(19) United States
(12) Patent Application Publication
(10) Pub. No.: US 2025/0115943 A1
(43) Pub. Date: Apr. 10, 2025

(54) SYSTEM AND METHOD FOR ANTIMICROBIAL SUSCEPTIBILITY TESTING

(71) Applicant: Fundamental Solutions Corporation, Easton, PA (US)

(72) Inventors: Joseph D. Kittle, JR., Easton, PA (US); Joel Lwande, Easton, PA (US)

(21) Appl. No.: 18/907,908

(22) Filed: Oct. 7, 2024

Related U.S. Application Data
(60) Provisional application No. 63/542,656, filed on Oct. 5, 2023.

(51) Int. Cl. C12Q 1/20

(52) U.S. Cl. CPC C12Q 1/20

(57) ABSTRACT

A method for performing antimicrobial susceptibility testing, comprising activating protein biosynthesis in microorganisms obtained from a biological sample in an acclimatization buffer; exposing the microorganisms to a library that includes various antimicrobials at predetermined concentrations, wherein exposure either kills the microorganisms or blocks protein biosynthesis in the microorganisms that are sensitive to one or more of the antimicrobials at one or more of the predetermined concentrations; labeling newly biosynthesized proteins produced by the microorganisms that survive exposure to the antimicrobials with a non-canonical amino acid (ncAA); tagging the labeled proteins with a detectable element attached to the ncAA, to create an amount of detectable signal; and comparing the amount of detected signal to a positive control, wherein an absence of or a decrease in the amount of signal relative to the positive control indicates effectiveness of one or more of the antimicrobials at one or more of the predetermined concentrations.

### CROSS-REFERENCE TO RELATED APPLICATIONS

This patent application claims the benefit of U.S. Provisional Patent Application Ser. No. 63/542,656 filed on Oct. 5, 2023 and entitled “System and Method for Antimicrobial Susceptibility Testing”, the disclosure of which is hereby incorporated by reference herein in its entirety and made part of the present U.S. utility patent application for all purposes.

### BACKGROUND

The disclosed inventive subject matter relates in general to systems, devices, and methods for use in diagnosing and treating infectious disease, and more specifically to a rapid antimicrobial susceptibility test for directly detecting susceptibility of various microorganism to various antimicrobials.

Selecting a proper antimicrobial (e.g., antibiotic) to treat a bacterial infection is typically accomplished through either polymerase chain reaction (PCR) identification of the bacteria and choosing a standard course of antibiotics or by directly testing antibiotic susceptibility to determine which antibiotics will inhibit the growth of the bacteria causing a specific infection. Bacteria may be identified with PCR; however, PCR does not directly confirm the susceptibility of the identified bacteria to a standard treatment regimen. Ineffective or incomplete treatment with antibiotics can lead to the development of antibiotic-resistant strains of bacteria, which is a widely recognized problem in modern healthcare. Direct antimicrobial susceptibility testing or antibiotic susceptibility testing (AST) may suggest a more successful treatment regimen, but such testing is typically much slower and more labor-intensive. Accordingly, there is an ongoing need for a high-throughput, rapid, reliable, and easy to use assay for directly determining the susceptibility of infectious microorganisms such as bacteria to a library of antimicrobials.

### SUMMARY

The following provides a summary of certain example implementations of the disclosed technology. This summary is not an extensive overview and is not intended to identify key or critical aspects or elements of the disclosed technology or to delineate its scope. However, it is to be understood that the use of indefinite articles in the language used to describe and claim the disclosed technology is not intended in any way to limit the described technology. Rather the use of “a” or “an” should be interpreted to mean “at least one” or “one or more”.

One implementation of the disclosed technology provides a first test method for determining the susceptibility of microorganisms to various antimicrobials, comprising activating protein biosynthesis in living microorganisms obtained from a native biological sample in an acclimatization buffer, wherein the acclimatization buffer is operative to activate the metabolism of the living microorganisms; exposing the living microorganisms to a library of antimicrobials, wherein the library of antimicrobials includes a plurality of antimicrobials at predetermined concentrations, and wherein exposure either kills the microorganisms or blocks protein biosynthesis in the microorganisms that are sensitive to one or more of the antimicrobials at one or more of the predetermined concentrations; labeling newly biosynthesized proteins produced by the living microorganisms that survive exposure to the antimicrobials by incorporating a non-canonical amino acid into the biosynthesized proteins; tagging the labeled proteins with a detectable element by attaching the detectable element to the non-canonical amino acid, wherein tagging the labeled proteins with the detectable element creates an amount of detectable signal; and detecting the signal and comparing the amount of detected signal to a positive control, wherein an observed absence of or a decrease in the amount of detectable signal relative to the positive control indicates effectiveness of one or more of the antimicrobials in the library of antimicrobials against the living microorganisms at one or more of the predetermined concentrations; and wherein an observed signal that approaches or is equal to the value of the positive control indicates ineffectiveness of one or more of the antimicrobials in the library of antimicrobials against the living microorganisms at one or more of the predetermined concentrations.

Certain implementations of the first test method further comprise using the absence of or decrease in detectable signal at a particular concentration of an effective antimicrobial to determine a minimum inhibitory concentration for each effective antimicrobial in the library of antimicrobials. Certain implementations of the test method further comprise using a wash buffer to remove any unincorporated non-canonical amino acid and using a wash buffer to remove any unattached detectable element, wherein one or both wash buffers contain a surfactant. In various implementations, the living microorganisms include bacteria, mycoplasmas, yeasts, fungal pathogens, protozoans, or combinations thereof. In one implementation, the native biological sample includes homogenized biopsy material that may include muscle, skin, or internal organs. In certain implementations. The native (i.e., direct from patient) biological sample may be taken directly from a bodily fluid, or the native biological sample may be an isolated colony cultured from a bodily fluid. In one implementation, the bodily fluid is urine. In other implementations, the bodily fluid is blood, sputum, synovial fluid, cerebrospinal fluid, saliva, breast milk, wound discharge fluid, ascites, semen, vaginal discharge, nasal mucus, or feces. In various implementations, the library of antimicrobials includes antibiotics, antifungals, or a combination thereof. Suitable antibiotics include beta-lactams, tetracyclines, aminoglycosides, macrolides, fluoroquinolones, sulfonamides, glycopeptides, oxazolidinones, ansamycins, lipopeptides, streptogramins, lincosamides, polymyxins, or combinations thereof. Suitable antifungals include azoles, echinocandins, polyenes, allylamines, flucytosine, griseofulvin, topical antifungals, or combinations thereof. The library of antimicrobials may also include bacteriophage. In one implementation, the non-canonical amino acid is homopropargylglycine (HPG), wherein the HPG includes an alkyne moiety, and wherein the newly biosynthesized proteins include the alkyne moiety. In one implementation, the detectable element is a fluorophore-tagged dye, wherein the fluorophore-tagged dye includes an azide group that reacts with the alkyne moiety of HPG. In other implementations, the detectable element is either an azide-modified biotin that reacts with the alkyne moiety of HPG, or an azido-conjugated enzyme that reacts with the alkyne moiety of HPG. In another implementation, the non-canonical amino acid is 3-Azido-L-alanine hydrochloride, wherein the 3-Azido-L-alanine hydrochloride includes an azide group, and wherein the newly biosynthesized proteins include the azide group. In another implementation, the detectable element is a fluorophore-tagged dye, and wherein the fluorophore-tagged dye includes an alkyne moiety that reacts with the azide group of 3-Azido-L-alanine hydrochloride. In certain implementations, the attachment of the detectable element to the labeled protein is accomplished using a copper catalysis that includes Copper I ions and a stabilizing ligand. The copper catalysis may be activated by addition of a reducing agent to a mixture of copper II ions and the stabilizing ligand, wherein the reducing agent is ascorbic acid, glyceraldehyde, or another reducing sugar such as aldose. In certain implementations, reagents used in the method are arranged in a kit that includes lyophilized buffers and lyophilized antimicrobials that exhibit prolonged shelf-life.

Another implementation of the disclosed technology provides a second test method for determining the susceptibility of microorganisms to various antimicrobials, comprising activating protein biosynthesis in living microorganisms obtained from an uncultured native biological sample taken directly from a bodily fluid in an acclimatization buffer, wherein the acclimatization buffer is operative to activate the metabolism of the living microorganisms; exposing the living microorganisms to a library of antimicrobials, wherein the library of antimicrobials includes a plurality of antimicrobials at predetermined concentrations, and wherein exposure either kills the microorganisms or blocks protein biosynthesis in the microorganisms that are sensitive to one or more of the antimicrobials at one or more of the predetermined concentrations; labeling newly biosynthesized proteins produced by the living microorganisms that survive exposure to the antimicrobials by incorporating a non-canonical amino acid into the biosynthesized proteins; tagging the labeled proteins with a detectable element by attaching the detectable element to the non-canonical amino acid, wherein tagging the labeled proteins with the detectable element creates an amount of detectable signal; detecting the signal and comparing the amount of detected signal to a positive control, wherein an observed absence of or a decrease in the amount of detectable signal relative to the positive control indicates effectiveness of one or more of the antimicrobials in the library of antimicrobials against the living microorganisms at one or more of the predetermined concentrations; and wherein an observed signal that approaches or is equal to the value of the positive control indicates ineffectiveness of one or more of the antimicrobials in the library of antimicrobials against the living microorganisms at one or more of the predetermined concentrations; and using the absence of or decrease in detectable signal at a particular concentration of an effective antimicrobial to determine a minimum inhibitory concentration for each effective antimicrobial in the library of antimicrobials.

Certain implementations of the second test method further comprise using a wash buffer to remove any unincorporated non-canonical amino acid and using a wash buffer to remove any unattached detectable element, wherein one or both wash buffers contain a surfactant. In various implementations, the living microorganisms include bacteria, mycoplasmas, yeasts, fungal pathogens, protozoans, or combinations thereof. In one implementation, the native biological sample includes homogenized biopsy material that may include muscle, skin, or internal organs. In certain implementations. The native (i.e., direct from patient) biological sample may be taken directly from a bodily fluid, or the native biological sample may be an isolated colony cultured from a bodily fluid. In various implementations, the bodily fluid is urine, blood, sputum, synovial fluid, cerebrospinal fluid, saliva, breast milk, wound discharge fluid, ascites, semen, vaginal discharge, nasal mucus, or feces. In various implementations, the library of antimicrobials includes antibiotics, antifungals, or a combination thereof. Suitable antibiotics include beta-lactams, tetracyclines, aminoglycosides, macrolides, fluoroquinolones, sulfonamides, glycopeptides, oxazolidinones, ansamycins, lipopeptides, streptogramins, lincosamides, polymyxins, or combinations thereof. Suitable antifungals include azoles, echinocandins, polyenes, allylamines, flucytosine, griseofulvin, topical antifungals, or combinations thereof. The library of antimicrobials may also include bacteriophage. In one implementation, the non-canonical amino acid is homopropargylglycine (HPG), wherein the HPG includes an alkyne moiety, and wherein the newly biosynthesized proteins include the alkyne moiety. In one implementation, the detectable element is a fluorophore-tagged dye, wherein the fluorophore-tagged dye includes an azide group that reacts with the alkyne moiety of HPG. In other implementations, the detectable element is either an azide-modified biotin that reacts with the alkyne moiety of HPG, or an azido-conjugated enzyme that reacts with the alkyne moiety of HPG. In another implementation, the non-canonical amino acid is 3-Azido-L-alanine hydrochloride, wherein the 3-Azido-L-alanine hydrochloride includes an azide group, and wherein the newly biosynthesized proteins include the azide group. In another implementation, the detectable element is a fluorophore-tagged dye, and wherein the fluorophore-tagged dye includes an alkyne moiety that reacts with the azide group of 3-Azido-L-alanine hydrochloride. In certain implementations, the attachment of the detectable element to the labeled protein is accomplished using a copper catalysis that includes Copper I ions and a stabilizing ligand. The copper catalysis may be activated by addition of a reducing agent to a mixture of copper II ions and the stabilizing ligand, wherein the reducing agent is ascorbic acid, glyceraldehyde, or another reducing sugar such as aldose. In certain implementations, reagents used in the method are arranged in a kit that includes lyophilized buffers and lyophilized antimicrobials that exhibit prolonged shelf-life.

Still another implementation of the disclosed technology provides a third test method for determining the susceptibility of microorganisms to various antimicrobials, comprising activating protein biosynthesis in living microorganisms obtained from either an uncultured native biological sample taken directly from a bodily fluid or an isolated colony cultured from a bodily fluid in an acclimatization buffer for a predetermined period of time, wherein the acclimatization buffer is operative to activate the metabolism of the living microorganisms; exposing the living microorganisms to a library of antimicrobials for a predetermined period of time; wherein the library of antimicrobials includes a plurality of antimicrobials at predetermined concentrations, and wherein exposure either kills the microorganisms or blocks protein biosynthesis in the microorganisms that are sensitive to one or more of the antimicrobials at one or more of the predetermined concentrations; labeling newly biosynthesized proteins produced by the living microorganisms that survive exposure to the antimicrobials by incorporating a non-canonical amino acid into the biosynthesized proteins; tagging the labeled proteins with a detectable element by attaching the detectable element to the non-canonical amino acid, wherein tagging the labeled proteins with the detectable element creates an amount of detectable signal; detecting the signal and comparing the amount of detected signal to a positive control, wherein an observed absence of or a decrease in the amount of detectable signal relative to the positive control indicates effectiveness of one or more of the antimicrobials in the library of antimicrobials against the living microorganisms at one or more of the predetermined concentrations; and wherein an observed signal that approaches or is equal to the value of the positive control indicates ineffectiveness of one or more of the antimicrobials in the library of antimicrobials against the living microorganisms at one or more of the predetermined concentrations; and using the absence of or decrease in detectable signal at a particular concentration of an effective antimicrobial to determine a minimum inhibitory concentration for each effective antimicrobial in the library of antimicrobials.

Certain implementations of the third test method further comprise using a wash buffer to remove any unincorporated non-canonical amino acid and using a wash buffer to remove any unattached detectable element, wherein one or both wash buffers contain a surfactant. In various implementations, the living microorganisms include bacteria, mycoplasmas, yeasts, fungal pathogens, protozoans, or combinations thereof. In one implementation, the native biological sample includes homogenized biopsy material that may include muscle, skin, or internal organs. In certain implementations. The native (i.e., direct from patient) biological sample may be taken directly from a bodily fluid, or the native biological sample may be an isolated colony cultured from a bodily fluid. In various implementations, the bodily fluid is urine, blood, sputum, synovial fluid, cerebrospinal fluid, saliva, breast milk, wound discharge fluid, ascites, semen, vaginal discharge, nasal mucus, or feces. In various implementations, the library of antimicrobials includes antibiotics, antifungals, or a combination thereof. Suitable antibiotics include beta-lactams, tetracyclines, aminoglycosides, macrolides, fluoroquinolones, sulfonamides, glycopeptides, oxazolidinones, ansamycins, lipopeptides, streptogramins, lincosamides, polymyxins, or combinations thereof. Suitable antifungals include azoles, echinocandins, polyenes, allylamines, flucytosine, griseofulvin, topical antifungals, or combinations thereof. The library of antimicrobials may also include bacteriophage. In one implementation, the non-canonical amino acid is homopropargylglycine (HPG), wherein the HPG includes an alkyne moiety, and wherein the newly biosynthesized proteins include the alkyne moiety. In one implementation, the detectable element is a fluorophore-tagged dye, wherein the fluorophore-tagged dye includes an azide group that reacts with the alkyne moiety of HPG. In other implementations, the detectable element is either an azide-modified biotin that reacts with the alkyne moiety of HPG, or an azido-conjugated enzyme that reacts with the alkyne moiety of HPG. In another implementation, the non-canonical amino acid is 3-Azido-L-alanine hydrochloride, wherein the 3-Azido-L-alanine hydrochloride includes an azide group, and wherein the newly biosynthesized proteins include the azide group. In another implementation, the detectable element is a fluorophore-tagged dye, and wherein the fluorophore-tagged dye includes an alkyne moiety that reacts with the azide group of 3-Azido-L-alanine hydrochloride. In certain implementations, the attachment of the detectable element to the labeled protein is accomplished using a copper catalysis that includes Copper I ions and a stabilizing ligand. The copper catalysis may be activated by addition of a reducing agent to a mixture of copper II ions and the stabilizing ligand, wherein the reducing agent is ascorbic acid, glyceraldehyde, or another reducing sugar such as aldose. In certain implementations, reagents used in the method are arranged in a kit that includes lyophilized buffers and lyophilized antimicrobials that exhibit prolonged shelf-life.

It should be appreciated that all combinations of the foregoing concepts and additional concepts discussed in greater detail below (provided such concepts are not mutually inconsistent) are contemplated as being part of the technology disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the descriptions provided herein are to be regarded as illustrative and not restrictive in nature.

### BRIEF DESCRIPTION OF THE DRAWINGS

The accompanying drawings, which are incorporated into and form a part of the specification, schematically illustrate one or more example implementations of the disclosed technology and together with the general description given above and detailed description given below, explain the principles of the disclosed subject matter, and wherein:

FIG. 1A-1B depict the CytoSPAR BLAST assay workflow, wherein FIG. 1A is a flowchart depicting an example stepwise process for completing the assay, and wherein FIG. 1B is a graphic comparing the stepwise process of FIG. 1A to a prior art clinical AST workflow.

FIG. 2 is a graphical representation of the click-chemistry reaction. The reaction requires the presence of copper in the reduced state. Sodium ascorbate reduces CU (II) to CU (I) and the chelator maintains copper in the reduced state.

FIG. 3 is an illustration of an example BLAST assay kit, wherein the kit contains an empty 2.0 L mixing bottle and buffers A, B, C1, C2, and W, as described in greater detail herein.

FIG. 4 is an illustration of an example CytoSPAR BLAST assay filter plate layout. In this implementation, all control wells (column 1) will not receive antibiotics, but will receive a tagging buffer (Buffer C1/C2 mix). Sterility control wells (column 1, rows A and B) will not receive bacteria, but will receive a labeling buffer (Buffer B). Positive control wells (column 1, rows C-F) will receive bacteria, a labeling buffer (Buffer B) and a tagging buffer (Buffer C1/C2 mix), but will not receive antibiotics. Background control wells (column 1, rows G and H) will receive bacteria and tagging buffer (Buffer C1/C2 mix), but will not receive labeling buffer (Buffer B) and antibiotics.

FIG. 5 is a flow diagram illustrating and describing an example process for converting BLAST AST assay data into reportable MIC values.

FIG. 6 is a graphic representation of contrived fluorescent plate reader data, wherein the upper panel represents a 96 well grid of fluorescence intensity readings, and wherein the lower panel depicts results from a 96 well plate used in traditional broth dilution analysis.

FIG. 7 is a graphical depiction of a first mathematical transformation of the test well values as described herein.

FIG. 8 is a graphical depiction of the second mathematical transformation of the test well values resulting in the generation of a “Truth Table” as described herein.

FIG. 9 is a graphical representation of an example determination of MICs involving looking for the first zero in the column of a “Truth Table” as described herein.

FIGS. 10A-10B are bar charts relating to testing fluorogenic and fluorescent dyes, wherein FIG. 10A is graphical representation of tagging labeled bacteria using different dyes, and wherein FIG. 10B is a graphical representation of the signal-to-noise ratio of each test relative to the background signal (negative control).

FIGS. 11A-11C depict BLAST antibiotic susceptibility test data for Gram-negative bacteria tested at 1.5×10⁸ CFUs/mL. FIG. 11A is a graphical representation of antibiotic susceptibility test using the disclosed BLAST assay for three strains of E. coli against nitrofurantoin: a susceptible strain (ATCC strain 25922); an intermediate strain (CDC strain 14); and a resistant strain (CDC strain 551). The data shown represents the average and standard deviations of 3 wells, with values for each assay relative to an antimicrobial-free control. FIG. 10B provides a graphical representation of the signal-to-noise ratio of each assay relative to the background signal (negative control). FIG. 10C provides a graphical representation of the Relative Response Ratio (RRR) showing relative labeling efficiency of the bacteria being tested. The fluorescent signal from FIG. 11A was transformed into RRR by subtracting the value of each test well from the positive control average and then dividing it by the value of the positive control average. Numbers close to zero indicate very little inhibition by the antibiotic and numbers close to one indicate a significant degree of inhibition.

FIGS. 12A-12C depict BLAST antibiotic susceptibility test for Gram-positive bacteria tested at 1.5×10⁸ CFUs/mL. FIG. 12A is a graphical representation of antibiotic susceptibility test using the BLAST assay for one strain of S. saprophyticus against nitrofurantoin: ATCC strain 15305. The data shown represents the average and standard deviations of 3 wells, with values for each assay relative to an antimicrobial-free control. FIG. 12B is a graphical representation of the signal-to-noise ratio of each assay relative to the background signal (negative control). FIG. 11C provides a graphical representation of the Relative Response Ratio (RRR) showing relative labeling efficiency of S. saprophyticus. The fluorescent signal from FIG. 12A was transformed into RRR by subtracting the value of each test well from the positive control average and then dividing it by the value of the positive control average. Numbers close to zero indicate very little inhibition by the antibiotic and numbers close to one indicate a significant degree of inhibition.

FIG. 13 is a bar chart depicting the results of a BLAST antibiotic susceptibility test for E. coli (ATCC strain #25922) against nitrofurantoin using 5×10⁵ CFUs/mL of bacteria. FIG. 13 provides a graphical representation of the Relative Response Ratio (RRR) showing relative labeling efficiency of the bacteria being tested. The data shown represents the average and standard deviations of 3 wells, with values for each assay relative to an antimicrobial-free control. The RRR values are calculated by subtracting the value of each test well from the positive control average and then dividing it by the value of the positive control average. Numbers close to zero indicate very little inhibition by the antibiotic and numbers close to one indicate a significant degree of inhibition. The concentration at which the RRR crosses the cutoff line represents the BLAST MIC (4 μg/mL in this case).

FIG. 14 is a bar chart depicting the results of a BLAST antibiotic susceptibility test for E. coli resistant strain (CDC, 551) against nitrofurantoin using 5×10⁵ CFUs/mL of bacteria. FIG. 14 provides a graphical representation of the Relative Response Ratio (RRR) showing relative labeling efficiency of the bacteria being tested. The data shown represents the average and standard deviations of 3 wells, with values for each assay relative to an antimicrobial-free control. The RRR values are calculated by subtracting the value of each test well from the positive control average and then dividing it by the value of the positive control average. Numbers close to zero indicate very little inhibition by the antibiotic and numbers close to one indicate a significant degree of inhibition. The concentration at which the RRR crosses the cutoff line represents the BLAST MIC (16 μg/mL in this case).

FIG. 15 is a bar chart depicting the results of a BLAST antibiotic susceptibility test for S. aureus (ATCC #29213) against nitrofurantoin using 5×10⁵ CFUs/mL of bacteria. FIG. 15 provides a graphical representation of the Relative Response Ratio (RRR) showing relative labeling efficiency of the bacteria being tested. The data shown represents the average and standard deviations of 3 wells, with values for each assay relative to an antimicrobial-free control. The RRR values are calculated by subtracting the value of each test well from the positive control average and then dividing it by the value of the positive control average. Numbers close to zero indicate very little inhibition by the antibiotic and numbers close to one indicate a significant degree of inhibition. The concentration at which the RRR crosses the cutoff line represents the BLAST MIC (4 μg/mL in this case).

FIGS. 16A-16B depict the results of a BLAST antibiotic susceptibility test for E. coli (ATCC strain #25922) against cefazolin using 5×10⁶ CFUs/mL of bacteria. FIG. 16A provides a graphical representation of the fluorescent signal of bacterial response to varying concentrations of cefazolin. The data shown represents the average and standard deviations of 3 wells, with values for each assay relative to an antimicrobial-free control. The positive control is bacteria labeled without antibiotics, while the negative control is bacteria without labeling and without antibiotics. FIG. 16B provides a table of the concentrations of cefazolin that were tested (column 1), and the Relative Response Ratio (RRR) showing relative labeling efficiency of the bacteria (column 2). Column 1 shows the CLSI QC range for cefazolin (1-4 μg/mL, shaded in grey) against E. coli (ATCC strain #25922). The fluorescent signal from FIG. 16A was transformed into RRR values (column 2) by subtracting the value of each test well from the positive control average and then dividing it by the value of the positive control average. Numbers close to zero indicate very little inhibition by the antibiotic and numbers close to one indicate a significant degree of inhibition. The test method used a cutoff value of 0.8 in the BLAST system and the concentration at which the RRR crosses the cutoff value represents the BLAST MIC. In this case the BLAST MIC is 2.0 μg/mL.

FIGS. 17A-17B depict the results of a BLAST antibiotic susceptibility test for E. coli (ATCC strain #25922) against doxycycline using 5×10⁶ CFUs/mL of bacteria. FIG. 17A provides a graphical representation of the fluorescent signal of bacterial response to varying concentrations of doxycycline. The data shown represents the average and standard deviations of 3 wells, with values for each assay relative to an antimicrobial-free control. The positive control is bacteria labeled without antibiotics, while the negative control is bacteria without labeling and without antibiotics. FIG. 17B provides a table of the concentrations of doxycycline that were tested (column 1), and the Relative Response Ratio (RRR) showing relative labeling efficiency of the bacteria (column 2). Column 1 shows the CLSI QC range for doxycycline (0.5-2 μg/mL, shaded in grey) against E. coli (ATCC strain #25922). The fluorescent signal from FIG. 17A was transformed into RRR values (column 2) by subtracting the value of each test well from the positive control average and then dividing it by the value of the positive control average. Numbers close to zero indicate very little inhibition by the antibiotic and numbers close to one indicate a significant degree of inhibition. The test method used a cutoff value of 0.8 in the BLAST system and the concentration at which the RRR crosses the cutoff value represents the BLAST MIC. In this case the BLAST MIC is 0.5 μg/mL.

FIGS. 18A-18B depict the results of a BLAST antibiotic susceptibility test for E. coli (ATCC strain #25922) against levofloxacin using 5×10⁶ CFUs/mL of bacteria. FIG. 18A provides a graphical representation of the fluorescent signal of bacterial response to varying concentrations of levofloxacin. The data shown represents the average and standard deviations of 3 wells, with values for each assay relative to an antimicrobial-free control. The positive control is bacteria labeled without antibiotics, while negative control is bacteria without labeling and without antibiotics. FIG. 18B provides a table of the concentrations of levofloxacin that were tested (column 1), and the Relative Response Ratio (RRR) showing relative labeling efficiency of the bacteria (column 2). Column 1 shows the CLSI QC range for levofloxacin (0.008-0.063 μg/mL, shaded in grey) against E. coli (ATCC strain #25922). The fluorescent signal from FIG. 18A was transformed into RRR values (column 2) by subtracting the value of each test well from the positive control average and then dividing it by the value of the positive control average. Numbers close to zero indicate very little inhibition by the antibiotic and numbers close to one indicate a significant degree of inhibition. The test method used a cutoff value of 0.8 in the BLAST system and the concentration at which the RRR crosses the cutoff value represents the BLAST MIC. In this case the BLAST MIC is 0.031 μg/mL.

FIGS. 19A-19B depict the results of a BLAST antibiotic susceptibility test for S. aureus (ATCC strain #29213) against cefazolin using 5×10⁶ CFUs/mL of bacteria. FIG. 19A provides a graphical representation of the fluorescent signal of bacterial response to varying concentrations of cefazolin. The data shown represents the average and standard deviations of 3 wells, with values for each assay relative to an antimicrobial-free control. The positive control is bacteria labeled without antibiotics, while negative control is bacteria without labeling and without antibiotics. FIG. 19B provides a table of the concentrations of cefazolin that were tested (column 1), and the Relative Response Ratio (RRR) showing relative labeling efficiency of the bacteria (column 2). Column 1 shows the CLSI QC range for cefazolin (0.25-1 μg/mL, shaded in grey) against S. aureus (ATCC strain #29213). The fluorescent signal from FIG. 19A was transformed into RRR values (column 2) by subtracting the value of each test well from the positive control average and then dividing it by the value of the positive control average. Numbers close to zero indicate very little inhibition by the antibiotic and numbers close to one indicate a significant degree of inhibition. The test method used a cutoff value of 0.8 in the BLAST system and the concentration at which the RRR crosses the cutoff value represents the BLAST MIC. In this case, the BLAST MIC is less than the lowest concentration tested (0.125 μg/mL).

FIGS. 20A-20B depict the results of a BLAST antibiotic susceptibility test for S. aureus (ATCC strain #29213) against doxycycline using 5×10⁶ CFUs/mL of bacteria. FIG. 20A provides a graphical representation of the fluorescent signal of bacterial response to varying concentrations of doxycycline. The data shown represents the average and standard deviations of 3 wells, with values for each assay relative to an antimicrobial-free control. The positive control is bacteria labeled without antibiotics, while negative control is bacteria without labeling and without antibiotics. FIG. 20B provides a table of the concentrations of doxycycline that were tested (column 1) and the Relative Response Ratio (RRR) showing relative labeling efficiency of the bacteria (column 2). Column 1 shows the CLSI QC range for doxycycline (0.125-0.5 μg/mL, shaded in grey) against S. aureus (ATCC strain #29213). The fluorescent signal from FIG. 20A was transformed into RRR values (column 2) by subtracting the value of each test well from the positive control average and then dividing it by the value of the positive control average. Numbers close to zero indicate very little inhibition by the antibiotic and numbers close to one indicate a significant degree of inhibition. The test method used a cutoff value of 0.8 in the BLAST system and the concentration at which the RRR crosses the cutoff value represents the BLAST MIC. In this case, the BLAST MIC is less than the lowest concentration tested (0.0625 μg/mL).

FIG. 21A-21B depict the results of a BLAST antibiotic susceptibility test for S. aureus (ATCC strain #29213) against levofloxacin using 5×10⁶ CFUs/mL of bacteria. FIG. 21A provides a graphical representation of the fluorescent signal of bacterial response to varying concentrations of levofloxacin. The data shown represents the average and standard deviations of 3 wells, with values for each assay relative to an antimicrobial-free control. The positive control is bacteria labeled without antibiotics, while negative control is bacteria without labeling and without antibiotics. FIG. 21B provides a table of the concentrations of levofloxacin that were tested (column 1) and the Relative Response Ratio (RRR) showing relative labeling efficiency of the bacteria (column 2). Column 1 shows the CLSI QC range for levofloxacin (0.063-0.5 μg/mL, shaded in grey) against S. aureus (ATCC strain #29213). The fluorescent signal from FIG. 21A was transformed into RRR values (column 2) by subtracting the value of each test well from the positive control average and then dividing it by the value of the positive control average. Numbers close to zero indicate very little inhibition by the antibiotic and numbers close to one indicate a significant degree of inhibition. The test method used a cutoff value of 0.8 in the BLAST system and the concentration at which the RRR crosses the cutoff value represents the BLAST MIC. In this case the BLAST MIC is 0.125 μg/mL.

FIGS. 22A-22B depict the results of a BLAST spiked urine versus isolate test results for levofloxacin-treated E. coli ATCC 25922 at 5×10⁵ CFU/mL. FIG. 22A is a chart depicting the fluorescent signal of bacterial response to varying concentrations of levofloxacin using both spiked urine samples and isolates. FIG. 22B is a chart depicting that the Relative Response Ratio (RRR) shows relative labeling efficiency of both spiked urine samples and isolates. The concentration at which the RRR crosses the cutoff value represents the BLAST MIC. In this case the BLAST MIC is 0.016 μg/mL for both spiked urine test and isolate test. The CLSI QC range for levofloxacin against E. coli is 0.008-0.06 μg/mL, breakpoint=0.5 μg/mL.

FIG. 23A-23B depict the results of a BLAST Fluorescent Response for aldose reduction catalysis. Three sugars (glyceraldehyde, glucose, and ribose) were tested using sodium ascorbate as the positive control and sucrose as the negative control. All tests were performed using E. coli ATCC 25922 at 5×10⁶ CFU/mL. FIG. 23A depicts the fluorescent signal of aldose-catalized BLAST assay; and FIG. 23B depicts the signal/noise ratio of the BLAST fluorescent signal.

FIG. 24 graphically depicts the effect of pH on aldose reduction catalysis in the BLAST assay. Different sugars were tested under different pH (7.4, 8.0, 9.0, and 10) conditions using E. coli ATCC 25922 at 5×10⁶ CFU/mL.

### DETAILED DESCRIPTION

Example implementations are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the disclosed technology. Accordingly, the following implementations are set forth without any loss of generality to, and without imposing limitations upon, the claimed subject matter.

The various embodiments and implementations disclosed and discussed herein are examples only and are provided to assist in the explanation of the apparatuses, devices, systems, and methods described herein. None of the features or components shown in the drawings or discussed below should be taken as required for any specific implementation of any of these apparatuses, devices, systems, or methods unless specifically designated as such. For ease of reading and clarity, certain components, modules, or methods may be described solely in connection with a specific Figure. Any failure to specifically describe a combination or sub-combination of components should not be understood as an indication that any combination or sub-combination is not possible. Also, for any methods described, regardless of whether the method is described in conjunction with a flow diagram, unless otherwise specified or required by context, any explicit or implicit ordering of steps performed in the execution of a method does not imply that those steps must be performed in the order presented but instead may be performed in a different order or in parallel.

In general, the disclosed technology provides a system and method for determining the susceptibility of various microorganisms to various antimicrobials. The method includes the generic steps of (i) activating protein biosynthesis in living microorganisms obtained from a native biological sample by transferring the living microorganisms to an acclimatization buffer; (ii) exposing the living microorganisms to a library of antimicrobials; (iii) labeling the newly biosynthesized proteins produced by the living microorganisms that survive exposure to the antimicrobial library with a non-canonical amino acid, wherein exposure of the microorganisms to the antimicrobials blocks protein biosynthesis in microorganisms sensitive to the antimicrobials and reduces the amount of labeled protein (thereby increasing the sensitivity of the assay); (iv) tagging the labeled proteins to create a detectable signal by attaching a detectable element to the non-canonical amino acid; (v) comparing the detectable signal to a positive control, wherein an observed decrease in detectable signal relative to the positive control indicates effectiveness of one or more antimicrobial in the library of antimicrobials against the living microorganisms; and (vi) using the decrease in detectable signal to determine a minimum inhibitory concentration for each effective antimicrobial in the library of antimicrobials. The terms “antibiotic” and “antimicrobial” are used interchangeably throughout this disclosure and it is to be understood that the term “antibiotic” may refer to both antibiotics and non-antibiotic antimicrobials, and that the term “antimicrobial” may refer to both non-antibiotic antimicrobials and antibiotics.

### The Blast Assay

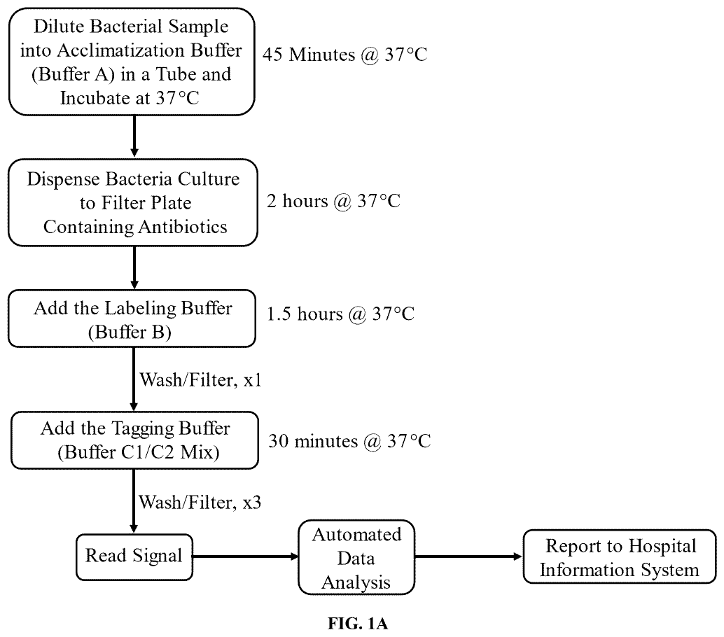

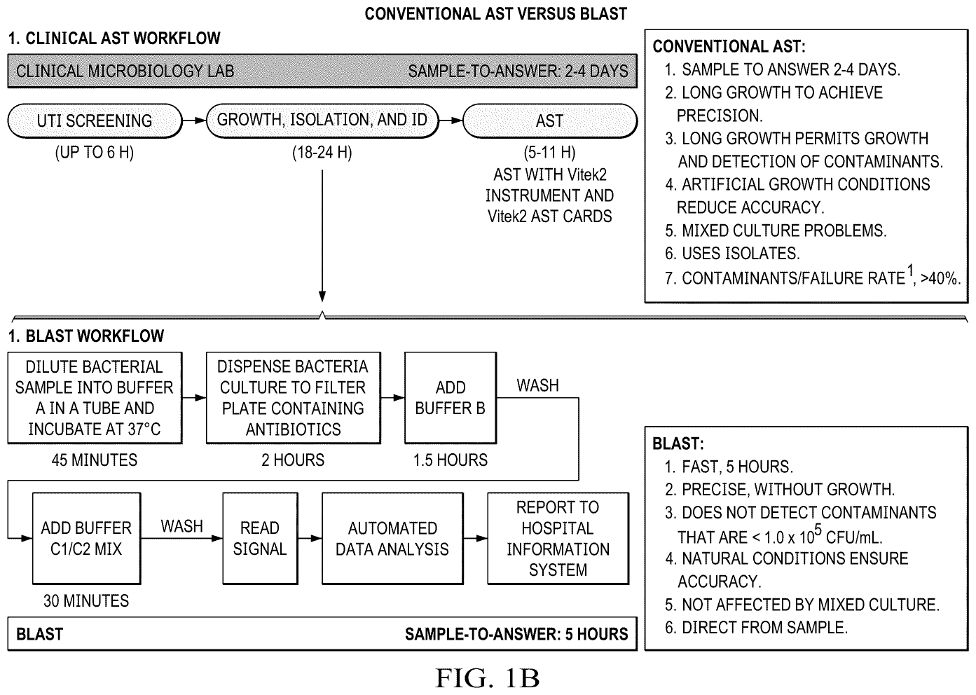

The disclosed technology, which is referred to as the CytoSPAR BLAST (Bacteria Labeling Antibiotic Susceptibility Test) system, is used to quantitatively assess bacterial minimal inhibitory concentration of antibiotics for in vitro susceptibility testing. Used as an aid in diagnosis for clinicians in determining potential treatment options for patients suspected of having a microbial infection, BLAST is intended to determine susceptibility of microorganisms to the listed antibiotics (or other antimicrobials) according to manufacturer's standards. The system is intended for use with clinical isolates from liquid culture and colonies grown on agar (solid medium) or directly from urine samples. Certain implementations of BLAST determine antibiotic susceptibility for a wide range of bacteria obtained from various types of patient samples, including samples from individuals suffering from urinary tract infections. The disclosed technology includes a high-throughput assay that directly detects bacterial susceptibility to a library of frequently used antibiotics and covers both Gram-negative and Gram-positive bacteria. As illustrated in FIG. 1A, the complete process can be summarized in four basic steps: (i) transfer of microorganisms to an acclimatization buffer to activate protein biosynthesis; (ii) antibiotic treatment and labeling; (iii) tagging; and (iv) signal readout.

An example embodiment of the CytoSPAR BLAST system provides a phenotypic (i.e., does not require foreknowledge of resistance genes involved or the mechanism of resistance) test that determines antibiotic susceptibility by detecting changes in the number of bacteria still living after incubation with a library of antibiotics. The test incorporates a non-canonical amino acid (ncAA) into newly produced proteins. The incorporated ncAA includes a reactive group which allows a specific modification and detection of the living bacteria. The assay involves bacterial incorporation of the ncAA, a chemical reaction between a reactive group and a fluorophore-tagged ligand, and detection of the newly fluorescent-tagged bacteria using a fluorescent plate reader or functionally similar device. The ncAA is added to bacterial growth media and is incorporated into newly synthesized bacterial proteins. The reactive groups on the ncAA are not naturally found in bacteria and act as a specific reactive group for bacteria undergoing active protein synthesis. The process is rapid with the ncAA being detectable in less than 2 hours after incubation (see FIG. 1B). The entire process is performed in a microtiter filter plate for high-throughput scale-up. Alternatively, a normal 96-well plate can also be used in which case centrifugation is used to wash the plate instead of filtration.

### Acclimatization, Antibiotic Treatment, Labeling, and Tagging

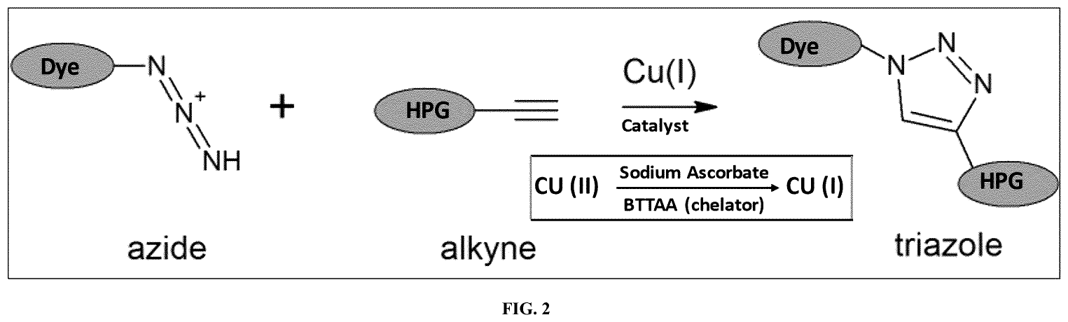

In an example embodiment, bacterial samples are first diluted and incubated in acclimatization media. After treatment with one or more antimicrobials, the ncAA is added and is taken up by living bacteria during protein biosynthesis in a process called labeling. As illustrated in FIG. 2, the newly synthesized proteins will have an alkyne group of the ncAA that will in turn react with an azide group found on a fluorophore-tagged ligand during the tagging process. The Click-Chemistry reaction between the alkyne group of the ncAA and the azide group of the fluorophore-tagged ligand requires the presence of copper in the reduced state-copper (I). In other implementations, the functional groups of the click chemistry reaction can be changed so that the ncAA has the azide group and the dye has the alkyne group. Sodium ascorbate is used in the reaction to reduce copper (II) to copper (I) and the chelator (BTTAA; Sigma-Aldrich #906326) maintains copper in a reduced state. Other reducing agents may be used in the BLAST assay, for example, aldoses (sugars) including glyceraldehyde, glucose, and ribose to replace sodium ascorbate. Likewise, alternate chelators may be used (e.g., histidine in place of BTTAA). In some implementations, metal-free click chemistry reactions are performed without using copper.

In various embodiments of the disclosed system, tagging of labeled bacteria is accomplished using either fluorogenic or fluorescent dyes that target the surface proteins and are compatible with the click chemistry reaction in solution. Commercially available fluorogenic (CalFluor 488 Azide; Click Chemistry Tools, #1369-1 and 3-Azido-7-hydroxycoumarin dye; Jena Biosciences, #CLK-FA047-1) and fluorescent (AZDye 488 Azide Plus dye; Click Chemistry Tools, #1475-25 and TideFluor 5WS Azide; AAT Bioquest, #2275) dyes were successfully used in the BLAST labelling process. Other modified versions of these dyes were developed by changing the wavelengths and other structures. Additional dyes have been used in the BLAST assay including: FastClick™ XFD488 Azide (AAT Bioquest, Cat. No. 72735), FastClick™ XFD555 Azide (AAT Bioquest, Cat. No. 72737), and iFluor 647 Azide Xtra (AAT Bioquest). Most of the dyes used contained azide groups for click chemistry coupling. In addition, labeling can also be performed using bright macromolecular dyes that were chemically conjugated to multiple azide groups. In another instance (Guy et al., 2022; Sarah et al., 2016), the dye (AZDye 488 DBCO; Click Chemistry Tools, #1278-1) had an alkyne group for coupling with the azide group present on the non-canonical amino acid (L-Azidohomoalanine; Click Chemistry Tools, #1066-25) substitute for methionine. This combination of L-Azidohomoalanine as the ncAA and AZDye 488 DBCO was different from the combination of HPG as the ncAA and the azide containing dyes because of the following two features: (i) The functional groups of the click chemistry reaction were changed; and (ii) the ncAA has the azide group and the dye has the alkyne group. The Click-Chemistry reaction is a metal-free reaction.

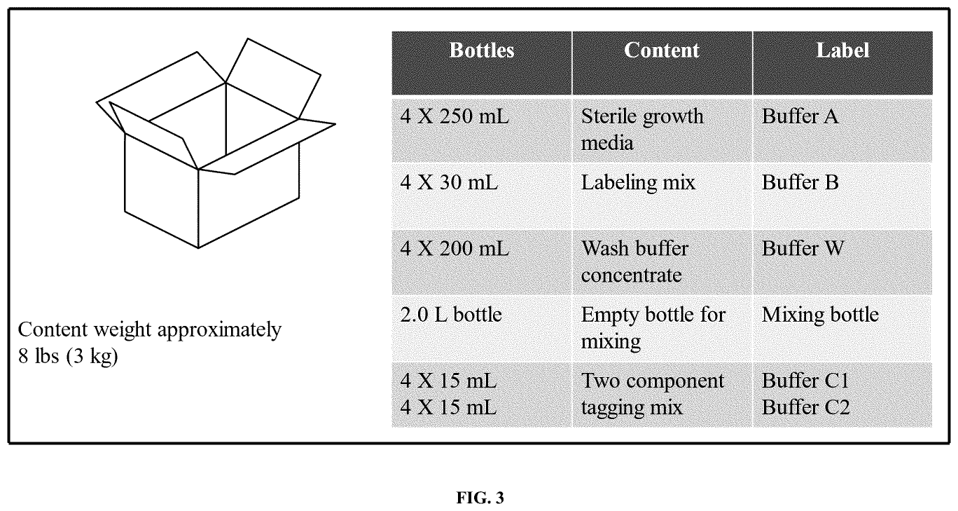

Using the BLAST system, tests can be performed on isolates or direct urine samples. Isolates may be from urine, blood, or other body fluids. Isolated bacteria may be either Gram-positive or Gram-negative. In an example implementation, living bacterial samples in acclimatization media (Buffer A) are distributed in a microtiter filter plate, wherein certain predetermined wells include antibiotics of interest at different concentrations. These antibiotics are arrayed with increasing concentrations in adjacent wells so that the bacterial response can be measured against each specific concentration. The response is measured quantitatively, and a value that corresponds to the ability of the bacteria to metabolize and grow is measured. The sample is first diluted in the acclimatization media (Buffer A), allowed to pre-incubate, and then distributed across an array of wells that include positive and negative controls and wells containing antibiotics. Positive control wells contain bacteria samples mixed with labeling buffer (Buffer B), but without antibiotics while negative control wells (background) are loaded with bacteria samples only, without antibiotics and without Buffer B. A few wells are incubated with Buffer A alone without bacteria samples to detect any contamination. Each 96 well filter plate can be used to test up to 11 different antibiotics against a bacterial sample. In some implementations, the BLAST assay is configured as a kit which includes a set of manufactured reagents distributed with the kit (see FIG. 3).

### Plate Design

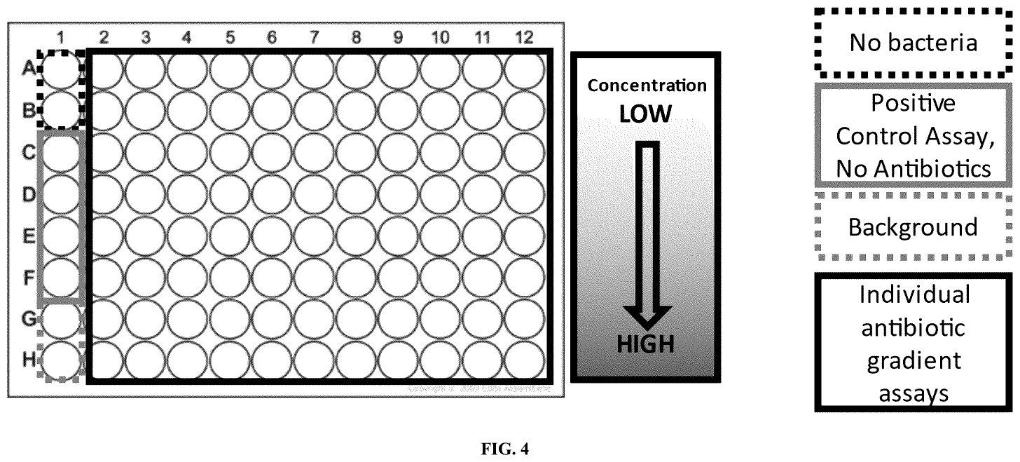

In the example plate layout shown in FIG. 4, eight (8) concentrations of each antibiotic are tested. The eight concentrations include the antibiotic breakpoint (the concentrations at which bacteria are susceptible to successful treatment with an antibiotic), four concentrations below the breakpoint and three concentrations above the breakpoint in two-fold dilution. Antibiotic breakpoints are set by the FDA and CLSI (see Reference 1, below). Following this design, up to eleven (11) antibiotics can be tested for each bacterial sample on a 96-well filter plate. One column of the plate is used for different controls.

### Data Processing and Analysis

Minimal Inhibitory Concentration (MIC) is defined as the lowest concentration of a chemical, usually a drug, which prevents visible growth of bacteria, fungi, or other microorganism of interest in vitro. MIC testing is used to determine an organism's susceptibility or resistance to an antibiotic or other antimicrobial. The antibiotic susceptibility test kit described herein is a comprehensive system that facilitates the determination of MIC values from BLAST Fluorescent plate readings.

### Data Management

Example implementations of the disclosed system employ a software data processing system that includes a spreadsheet model for converting BLAST test data into reportable MIC values. The system is designed to meet FDA requirements and expectations, and fits well with the predicate and gold standard practices. The data processing system corrects for background and adjusts for sample-specific variability in labeling, thereby improving the accuracy of the MIC values. Additionally, the system allows for the inclusion of experimentally determined parameters for each antibiotic, providing enhanced accuracy and precision in the determination of

MIC values. The system is designed to minimize operator keystrokes or data entry, reducing the potential for human error, and improving efficiency. Furthermore, the data processing system can convert fluorescent plate reader .CSV files to MIC values, providing a complete approach to MIC determination. With this antibiotic susceptibility test kit, users can confidently determine MIC values, aiding in the selection of appropriate antibiotic therapies and promoting the effective treatment of bacterial infections. Certain implementations of this system capture meta data such as date, kit version and lot information and test-specific information including test operator ID and sample ID information. The software methodology described below can be implemented on a variety of platforms including as a spreadsheet, a series of scripts in a database, or as stand-alone coded software.

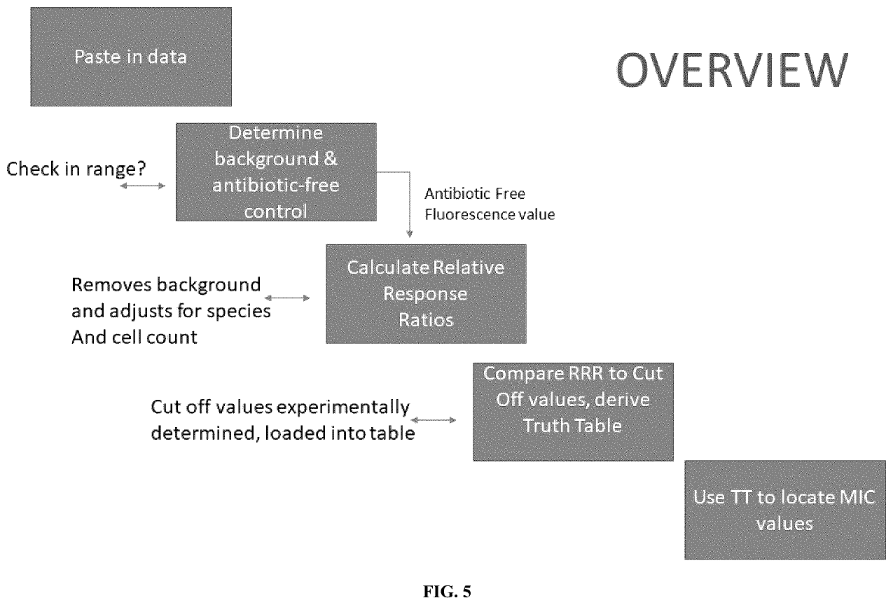

The .CSV file is copied from the fluorescent plate reader to the software (spreadsheet) in the designated space. The spreadsheet then processes the input along with information specific to the test kit composition (antibiotic identities, concentrations etc.) and the results appear on the results page. The spreadsheet takes the information through a series of steps where each is individually accessible, as depicted in the flow diagram of FIG. 5.

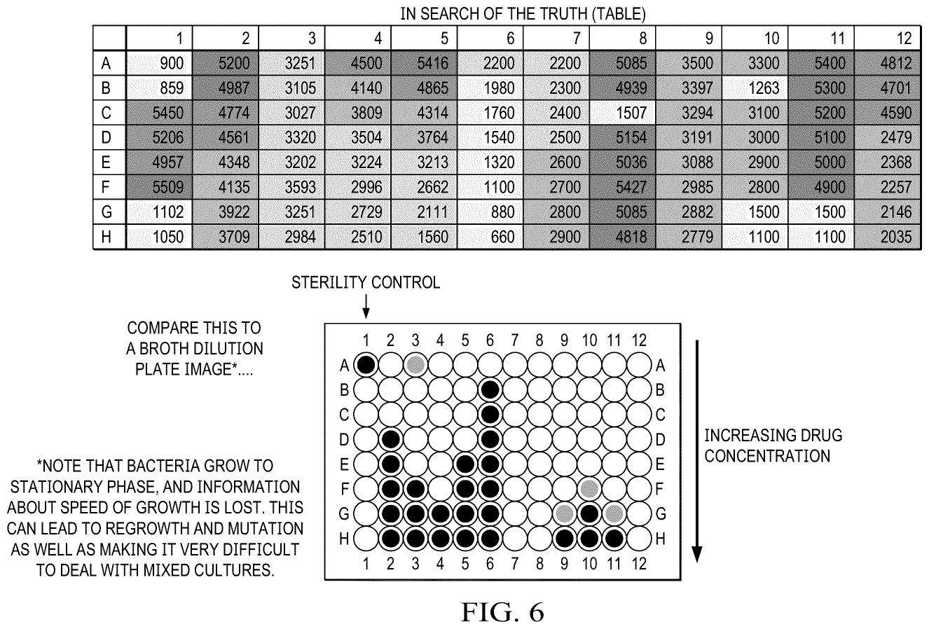

In an example embodiment, the BLAST analysis is based on a 96 well plate configuration that contains both controls and a library of antibiotics for treating bacteria to determine susceptibility to the antibiotics. The antibiotic library is supplied from a 96 deep-well microtiter plate preloaded with compounds ready to reconstitute at stock concentrations. The library is dispensed by the user into an array of concentrations in defined locations on the 96 well filter plate as depicted in FIG. 4. The processing of a bacterial sample results in wells that exhibit varying levels of fluorescence, depending on the activity of the antibiotic against the bacteria being tested. A contrived example for this output table is shown in the upper panel of FIG. 6. In the lower panel of the same Figure, there is a comparison image of results from a 96 well plate used in traditional broth dilution analysis, which also uses an array of antibiotics at various concentrations, and which is read visually by a human operator. Wells that have effective antibiotics at proper concentration prevent bacterial growth. In the BLAST 96 well filter plate assay system, the assay arrives at the result faster, and wells where the bacteria are inhibited exhibit lower fluorescence compared to control wells or where bacteria are resistant to the antibiotic.

Processing the Data from the Control Wells

The first data processing step involves obtaining averages for the values in the two sets of controls. The first control is the sample bacteria processed without any antibiotic, which should exhibit a high degree of labeling (positive control). Averaging these wells shown in FIG. 4 and FIG. 6 as wells in column 1 rows C, D, E, and F provides an average of uninhibited fluorescent value. The other control is wells with bacteria grown without adding the labeling buffer and antibiotics (background) but still processing all the other steps (wells in column 1 rows G and H). This provides a good estimate of the background caused by non-specific labeling and the plate itself. Average values for each of these controls are compared to the expected range for these samples, and if it is out of range, the software indicates that the assay has failed. The positive control average should also be higher than the value of the background control, by at least some predetermined limit (for example, at least 30% greater). Otherwise, the signal to noise ratio is too small to return a valid response. Finally, the sterility (no added bacteria) controls (wells in column 1 rows A and B) are processed and must stay below a predetermined threshold value to ensure that the plate or any of the reagents are not grossly contaminated.

Processing the Data from the Antibiotics Array Wells

Each well in column 2-12 represents a portion of the bacteria treated with some predetermined concentration of an antibiotic. If the antibiotic inhibits cell metabolism or kills the cell, the labeling will be reduced, if the concentration of antibiotic fails to significantly inhibit the bacteria, the bacteria will label as well, or nearly as well, as the positive control (uninhibited wells). Given the nature of the labeling and tagging process, even an antibiotic that is clinically effective may allow some measurable amount of labeling in the sample. This is not a negative, even bacteria that fail to grow well in the traditional test have the potential to be effectively labeled in the BLAST test. What is relevant is that the labeling observed is above the signal to noise cut off, and that the effective antibiotic at the right concentration significantly inhibits this labeling. Replication of the bacteria is neither necessary nor required during the disclosed assay/test.

### First Mathematical Transformation of the Test Well Values

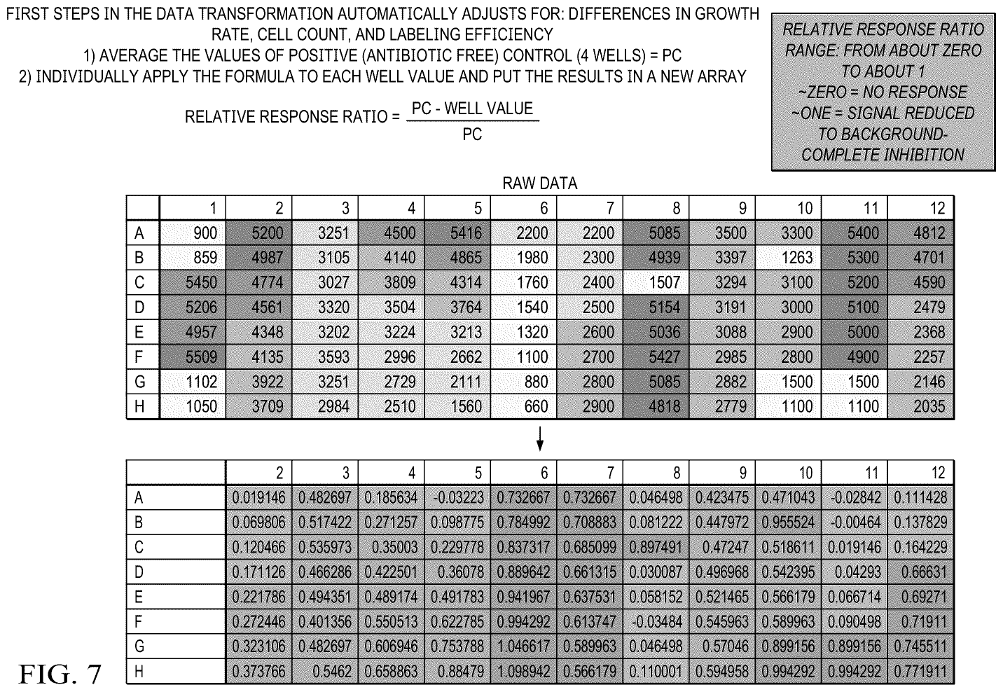

The method used to process the data must account for residual labeling, and it must also automatically adjust to the overall efficiency of bacterial labeling, which can vary depending on the type, and concentration and condition of bacteria entering the test. In the described system and method (see FIG. 7), the difference between the positive control average and the value for each well is computed (positive control average-value for the well). This difference is divided by the value of the positive control average (the divisor is the average of the uninhibited bacteria values). This effectively takes into account that different samples exhibit different levels of labeling efficiency and typically returns a number close to the range of zero to one. Numbers close to zero indicate very little inhibition by the antibiotic and numbers close to one indicate a significant degree of inhibition. In some implementations, the divisor may be the positive control value minus the average background value. This has the effect of increasing the size of the overall number, making the test somewhat less sensitive, but also less subject to noise variations. The mathematical function calculates a value which is referred to as the “Relative Response Ratio” or RRR. The RRR is calculated for each well value of the array and represents a map of the values normalized for the relative labeling efficiency of the bacteria being tested.

### Second Mathematical Transformation of the Test Well Values

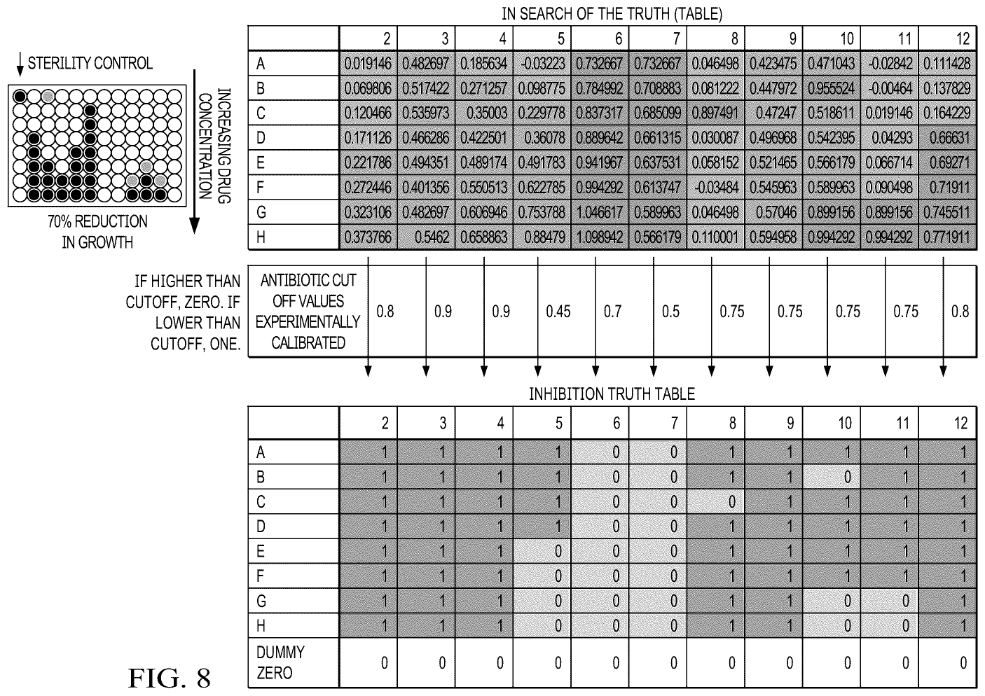

The next data processing step is important to both process the data to a recognizable output but also to allow for a calibration of the assay for differences in the way the samples are expected to respond to different antibiotics. In this step (see FIG. 8), the data in the array is converted to what is called a truth table using supplied cut off values that are calibrated using experimental data (a typical cut off value would be 0.7, but there might be slight variations). Values near the numerical value of 1 are converted to a value of zero and values near zero are converted to the number 1. One simple way to do this is to use a logic statement, if/then. If the RRR value from the table is higher than the cut off, then the logic statement returns a value of zero, if the number is lower than the cut off, then the value of 1 is returned. The RRR is a measure of growth compared to the controls so if that growth is close to the control, then 1 represents that the cells are efficiently labeling. If the compound in the array is inhibiting growth at a specific concentration, then zero represents this reduced growth. In this assay, the array of values in the truth table (zeros and ones) is reminiscent of the appearance of a traditional broth plate with growth or non-growth based on concentration. A key point is that the cut off value can be calibrated using experimental data, so that the truth table corresponds to authentic inhibition within the time frame and under the conditions of the bacterial labeling assay. In other implementations, the zero and ones are represented by other symbols or colors, as long as they are defined within the system.

Using zeros and ones facilitates the next two steps in the analysis. It is common in this type of analysis for some small percentage of wells to fail. This typically looks like an isolated well in a series of concentrations of a particular antibiotic having an anomalously high or low value. In the Industry Standards Guidance, when these wells are encountered in the traditional broth dilution analysis, they are scored according to the values of adjacent wells based on a set of rules. For example, if bacteria fail to grow in one well then it is scored as inhibited only if the next well at a higher concentration are also not growing. With regard to the disclosed system, if a well is scored zero, it is a valid result if the next higher concentration well is also scored as zero.

### Determination of MICs

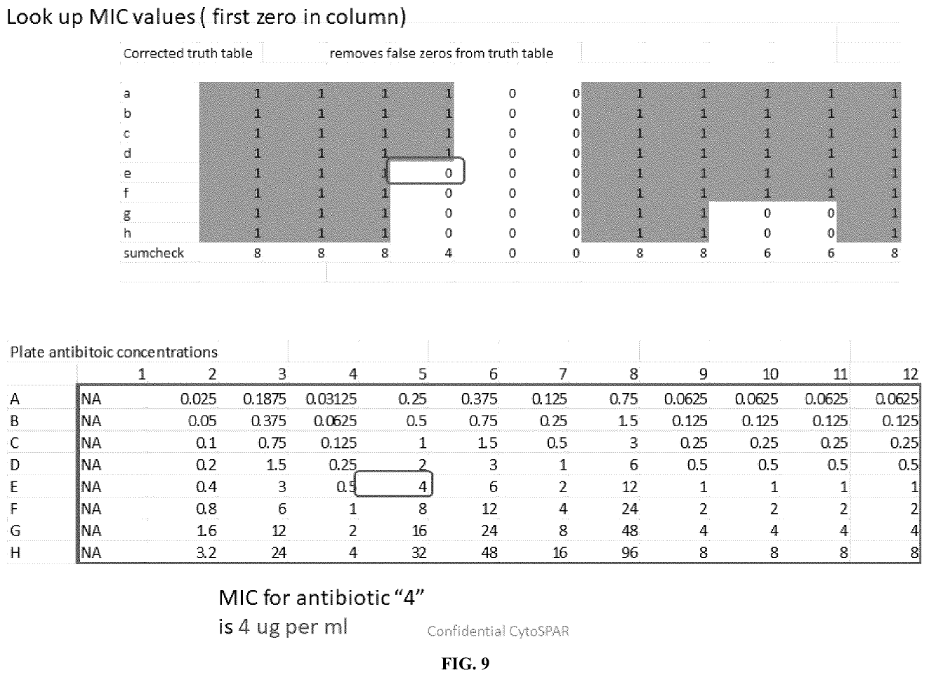

Once anomalous values are removed from the table, it is then possible to score the response of the bacteria being tested to each antibiotic (see FIG. 9). For each 96 well plate processed, there is a map of the corresponding antibiotics and their concentrations at each well location. The lowest concentration of antibiotic that effectively inhibits bacterial growth is called the “minimal inhibitory concentration” or MIC. Thus, the zero in the table corresponds to wells where the concentration is effective at inhibiting the bacterial metabolism to a level below the predetermined threshold value. A simple function to look up the lowest concentration of each antibiotic that reaches the threshold value (a zero in the table, in this example) identifies the MIC for that antibiotic. In many cases, the bacteria may be able to grow at all concentrations of a particular antibiotic tested, in which case the software (all the values in the column are “1”), returns the value “resistant to greater than the top value tested”. Conversely, if the bacteria are sensitive to all the concentrations tested then the value is “sensitive to less than the lowest value tested”. The results are then used to populate a report that includes the MIC of each antibiotic tested with regard to a particular sample.

### Blast Assay Materials, Reagents, and Equipment

In an example implementation, the BLAST assay includes use of the materials, reagents, and equipment listed below in TABLE 1.

TABLE 1
Materials, reagents, and equipment for the BLAST assay.

| Component | Purpose | Vendor | Part Number |
|---|---|---|---|
| The BLAST kit | Assay buffers | CytoSPAR | NA |
| SpectraMax M2 Plate Reader | Signal readout | Molecular | — |
| | | Devices | |
| Antibiotics | AST assay | CytoSPAR | |
| Vacuum manifold and Pump (Wel- | Plate filtration | Southern | 195200-11 |
| Vac 200, Microplate Vacuum | | labware | |
| Manifold, with Rocker 300) | | | |
| 96 well filter plates | Assay performance | PALL | 8019 |
| 96 well plate seal | Sealing the bottom of the | VWR | 60941-064 |
| | assay plate | | |
| AeraSeal plate adhesive, sterile | Sealing the top of the | VWR | 490007-322 |
| | assay plate | | |
| Spreadsheet and Spreadsheet | Data processing and | CytoSPAR | NA |
| support package | analysis | | |
| Blood agar plates | Preparation of isolates | NA | NA |
| Shaker/Incubators (96 well plate | Incubating the plate at | NA | NA |
| shaker) (Labnet Orbit P4 Digital | 37° C. with shaking at 250 | (multiple | |
| Shaker) | rpm. | vendors) | |

Additional materials used with the assay include: a pipet (P300) and filtered pipet tips; sterile water (water for irrigation) or equivalent; a multi-channel pipette capable of pipetting 20-300 uL; a timer; 15 mL and 50 mL culture tubes; a vortex mixer; control bacteria for system validation (e.g., E. coli, ATCC 25922); a multi-channel reagent reservoir; a biosafety hood; a densitometer; and McFarland standards. In alternate implementations, regular 96-well plates are used in the assay, in which case a centrifuge is used for the wash step.

### The Blast Assay Kit

In an example implementation, the BLAST assay kit (see FIG. 3) contains an empty 2.0 L mixing bottle (labeled with a fill mark of 2.0 L), and five buffers labelled A, B, C1, C2, and W. One kit includes reagents sufficient for testing 48 samples using 48 filter/regular plates. Each sample can be tested against up to 11 antibiotics on a single 96-well filter/regular plate. Buffer A is an acclimatization media; Buffer B is a labeling buffer; Buffer C1 and C2 are a tagging buffer mix; and Buffer W is a wash buffer. Regarding the level of precision needed for each solution, the targeted concentrations, amounts, and volumes should be within +/−5% of target. Samples that do not fall within 10% of target should be rejected.

In an example implementation, Buffer A, which is the acclimatization buffer, contains Brain Heart Infusion (BHI) (Sigma #53286). In other implementations, Buffer A is prepared from Muller Hinton Media or YPD (e.g., for yeast testing). With regard to the contents of an example kit for the BLAST assay, 1.0 L of Buffer A is packaged in 250 mL×4 bottles per kit, with each bottle sufficient for testing 12 plates. Buffer A is prepared based on the example formulation described in TABLE 2, below.

In an example implementation, Buffer B, which is the labelling buffer solution, contains HPG (Click Chemistry Tools, #1067-25) in sterile water as described in TABLE 2. With regard to the contents of an example kit for the BLAST assay, 120 mL of Buffer B is packaged in 30 mL×4 bottles per kit, with each bottle sufficient for testing 12 plates.

In an example implementation, Buffer C1 (see TABLE 2), which is the first component of a 10× tagging buffer solution mix, includes CuSO₄, AZDye 488 Azide, and BTTAA in 10×HEPES Buffered Saline with Tween20. 10×HEPES Buffered Saline with Tween20 includes HEPES free acid, NaCl, pH 7.4, and Tween20. 60 mL of Buffer C1 (10× concentrate in 10×HEPES buffer) is packaged in 15 mL×4 bottles per kit, with each bottle sufficient for 12 plates. In an example implementation, Buffer C2 (see TABLE 2), which is the second component of a 10× tagging buffer solution mix, includes Sodium ascorbate in sterile water. With regard to the contents of an example kit for the BLAST assay, 60 mL of Buffer C2 (10× concentrate in sterile water) is packaged in 15 mL×4 bottles per kit, with each bottle sufficient for 12 plates.

In an example implementation, Buffer W (see TABLE 2), which is a 10× wash buffer solution, includes 10×PBS with Tween20. With regard to the contents of an example kit for the BLAST assay, 800 mL of Buffer W (10× concentrate) is packaged in 200 mL×4 bottles with each bottle sufficient for 12 plates. This 10×PBS buffer includes NaCl, KCl, Na₂HPO₄, KH₂PO₄, and Tween20, pH 7.4. The 2.0 L mixing bottle is used to make a 1× solution of wash buffer.

TABLE 2
BLAST Assay Buffers

| Buffer | Component | Concentration range | |
|---|---|---|---|
| A (1X) | BHI | 17.5-40 | g/L |
| | or Muller Hinton Media | 20-40 | g/L |
| | or YPD (for yeast) | | |
| | Peptone | 20 | g/L |
| | Yeast extract | 10 | g/L |
| | Glucose | 20 | g/L |
| B (1X) | HPG (chemical name) | 0.5-50 | mg/mL |
| C1 (10X concentrate) | Copper II sulfate | 50-1000 | μM |
| | Ligand | 20-200 | mg/L |
| | BTAA or TBA or histidine | | |
| | Surfactant | 1-5% | |
| | Tween 20 | | |
| | AZDye 488 Azide Plus | 50-150 | mg/L |
| C2 (10X concentrate) | Sodium Ascorbate | 2-10 | g/L |
| | or Glyceraldehyde | | |

| | | |
|---|---|---|
| W (10X concentrate) | NaCl | 0.75-2M |
| | Sodium Phosphate or | |
| | HEPES pH 7 to 8 | |
| | Surfactant (Tween 20) | 1-5% |

For conferring stability to the disclosed reagents, the acclimatization media (Buffer A) and the wash buffer (Buffer W) are stored at room temperature; the labeling buffer (Buffer B) is stored at room temperature; the tagging buffer mix (Buffers C1 and C2) are stored at −20° C.; and the antibiotics supplied with the assay kit are stored at 4° C. For long term stability, the tagging buffer mix (Buffers C1 and C2) may be lyophilized and stored at room temperature. The antibiotics may also be lyophilized and supplied as lyobeads in the BLAST kit for long term stability at room temperature.

Initially, when performing the BLAST Assay, the SpectraMax M2 plate reader (or other suitable device), is powered on and the SoftMax Pro 7.1 software is opened. “Start a new plate” is selected in the SoftMax Pro 7.1 software with the SpectraMax Pro 7.1 settings being: (a) Read mode: Fluorescence; (b) Read type: Endpoint; (c) Wavelengths: Excitation 484 nm; Emission 524 nm; (d) Plate type: 96 well standard opaque; (e) PMT and Optics: Auto (6 flashes per read); (f) Shake: Off; and (g) Read area: Based on the assay design and wells in use.

In an example implementation, 96 well filter plates for the assay are supplied together with a 96 deep well plate containing respective antibiotic solutions. Each antibiotic is supplied at a concentration that is 2× the highest assay concentration based on the assay design. Alternatively, antibiotics can be supplied in form of lyobeads that are dissolved in sterile water immediately before use. Filtration/washing is done by using the vacuum pump/manifold with the pressure set at 20-25 mm Hg for 2 minutes. The filter plates are sealed at the bottom using an adhesive film and at the top using the AeraSeal plate adhesive for 96 well assay plates during the incubation steps. Both seals are removed during the wash/filtration steps.

An example implementation of the BLAST assay includes three controls that are run on the 96-well filter/regular plate (see FIG. 4) along with different antibiotic concentrations. The three controls are a sterility control (no bacteria, only Buffer A, Buffer B, and Buffer C1/C2 mix are added to the respective wells without antibiotics); a positive control (bacteria is treated with Buffer B and Buffer C1/C2 mix without antibiotics); and a negative control/background (bacteria is treated with Buffer C1/C2 mix without Buffer B and without antibiotics). TABLE 3 below provides a description of the BLAST assay controls. The three controls are incorporated into the assay on the 96-well filter/regular plate as follows: sterility control (column 1, rows A and B); positive control (column 1, rows C-F); background (column 1, rows G and H).

TABLE 3
BLAST Assay Controls.

| | Buffer | Bacteria | Anti- | Buffer | Buffer |
|---|---|---|---|---|---|
| Type of control | A | sample | biotics | B | C1/C2 mix |
| Sterility control (no | + | − | − | + | + |
| bacteria, no | | | | | |
| antibiotic) - column | | | | | |
| 1, rows A and B | | | | | |
| Positive control | + | + | − | + | + |
| (labeled and tagged | | | | | |
| bacteria without | | | | | |
| antibiotics) - column | | | | | |
| 1, rows C-F | | | | | |
| Background (tagged | + | + | − | − | + |
| bacteria without | | | | | |
| labeling and without | | | | | |
| antibiotics) - column | | | | | |
| 1, rows G and H | | | | | |

The BLAST assay can be performed on a 96-well filter plate or on a regular 96-well plate. When a 96-well filter plate is used in the assay, the wash steps are accomplished by filtration. However, when a regular 96-well plate is used in the assay, the wash steps are accomplished by centrifugation. The following protocol describes the BLAST assay as performed on a 96-well filter plate with the wash steps accomplished by filtration.

### Blast Assay Protocol

STEP 1: Determine the amount of buffer needed for the assay and remove Buffers C1 and C2 from −20° C. storage to equilibrate to room temperature. Testing 12 samples on 12 filter plates will require: 250 mL×1 bottle of Buffer A; 30 mL×1 bottle of Buffer B; 15 mL×1 bottle of Buffer C1; 15 mL×1 bottle of Buffer C2; and 200 mL×1 bottle of Buffer W.
STEP 2: To test from isolates, pick different colonies from a blood agar plate and inoculate 10 mL of Buffer A in a 50 mL culture tube at 5.0×10⁶ CFU/mL (use the 0.5 McFarland standard and then dilute to start the BLAST assay at 5.0×10⁶ CFU/mL). Incubate the culture at 37° C. for 45 minutes with shaking at 250 rpm. To test from a direct urine sample, centrifuge the urine sample, discard the supernatant and resuspend the pellet in 10 mL of Buffer A in a 50 mL culture tube. Incubate the culture at 37° C. for 45 minutes while shaking at 250 rpm. For direct urine samples, up to 30 mL of patient urine is centrifuged to collect the putative bacteria and then resuspended in Buffer A to a concentration of between 1×10⁵ CFU/mL to 5×10⁶ CFU/mL as judged by turbidity measurements. Incubate the culture for 45 minutes with shaking at 37° C. degrees as above. For a lower concentration range, increase the incubation time to 60-90 minutes.
STEP 3: During bacterial incubation in STEP 2, remove the antibiotic stocks from the 4° C. storage and allow a 20-minute room temperature equilibration. Use the plate seal to cover the bottom of the 96-well filter plate and prepare the antibiotic dilutions in the plate using a serial dilution (described below). Use a multi-channel pipet to add 65 μL of Buffer A into each well of the filter plate. Use a multi-channel pipet to transfer 65 μL of the supplied antibiotics from the rows of the deep well plate into corresponding wells in row H, columns 2-12 of the filter plate. Mix by pipetting 4 times (this is the highest concentration of each antibiotic in the assay) and then proceed to serial dilution. Serial Dilution Method: On the 96-well filter/regular plate, transfer 65 μL of the antibiotic mix from wells in columns 2-12, row H into respective wells in columns 2-12, row G and mix by pipetting 4 times. Continue with this dilution sequence until row A. Remove and discard the extra 65 uL from wells in row A, columns 2-12.
STEP 4: To prepare the bacteria culture from step 2, invert, mix and vortex the culture thoroughly before plating to maintain uniformity. Transfer the culture from the tube to the 25 mL sterile reagent reservoir for loading with the multi-channel pipet. Add 65 μL of bacteria culture from step 2 into each well of columns 2-12 of the filter plate using a multi-channel pipet. Controls: (a) Sterility control (column 1, rows A and B). Add 65 μL of Buffer A into each well of column 1, rows A and B of the filter plate; (b) Positive control (column 1, rows C-F). Add 65 μL of bacteria culture from step 2 into each well of column 1, rows C-F of the filter plate; (c) Background (column 1, rows G and H). Add 65 μL of bacteria culture from step 2 into each well of column 1, rows G and H. Cover the plate with the AeraSeal plate adhesive and incubate at 37° C. for 2 hours while shaking at 250 rpm.
STEP 5: Remove the AeraSeal and add 20 μL of Buffer B to each well of columns 2-12 of the filter plate using a multi-channel pipet. Controls: (a) Sterility control (column 1, rows A and B). Add 20 μL of Buffer B into each well of column 1, rows A and B of the filter plate; (b) Positive control (column 1, rows C-F). Add 20 μL of Buffer B into each well of column 1, rows C-F of the filter plate; (c) Background (column 1, rows G and H). Add 20 μL of sterile water into each well of column 1, rows G and H. Cover the plate with the AeraSeal plate adhesive and incubate at 37° C. for 1.5 hours while shaking at 250 rpm. Note: During the incubation in step 5, dilute Buffer W to 1× concentration by adding 200 mL of the buffer into the supplied 2 L mixing bottle. Add sterile water up to the 2 L mark (1.8 L) and mix. For smaller quantities, mix Buffer W with sterile water in a ratio of (1 Buffer W:10 sterile water).
STEP 6: Remove the AeraSeal plate adhesive from the top and the seal from the bottom of the filter plate. Drain out the liquid by filtering the 96-well filter plate using a vacuum manifold. Wash the plate by adding 300 μL of diluted Buffer W into each well and filter the plate using a vacuum manifold. Discard the filtrate (flow-through). Perform the filtration step for 2 minutes using the vacuum pump/manifold with the pressure set at 20-25 mm Hg (the plate should be visibly drained). Use the plate seal to cover the bottom of the filter plate.
STEP 7: For 12 filter plates, mix 15 mL of Buffer C1 and 15 mL of Buffer C2. Add 120 mL of sterile water and mix well. Add 100 μL of the Buffer C1/C2 mix into each well of the filter plate. Cover the plate with the AeraSeal plate adhesive and incubate at 37° C. for 30 minutes while shaking at 250 rpm. For 1 filter plate, mix 2 mL of Buffer C1 and 2 mL of Buffer C2. Add 16 mL of sterile water and mix well. Add 100 μL of the Buffer C1/C2 mix into each well of the filter plate. Cover the plate with the AeraSeal plate adhesive and incubate at 37° C. for 30 minutes while shaking at 250 rpm.
STEP 8: Remove the AeraSeal plate adhesive from the top and the seal from the bottom of the filter plate. Drain out the liquid by filtering the 96-well filter plate using a vacuum manifold. Wash the plate by adding 300 μL of diluted Buffer W to each well and filter the plate using a vacuum manifold. Repeat the wash step twice and discard the filtrate (flow-through). Perform each filtration step for 2 minutes using the vacuum pump/manifold with the pressure set at 20-25 mm Hg (the plate should be visibly drained). After the final wash, clean the plate to remove any liquid drops adhering to the bottom of the plate using a paper towel and proceed to STEP 9.
STEP 9: Record the endpoint fluorescence of the filter plate after the final wash using the SpectraMax M2 plate reader (excitation 484 nm, and emission 524 nm, using auto PMT settings).

Data Processing and Analysis of the BLAST Assay includes the following steps:
1. Copy the .CSV file from the fluorescent plate reader to the provided spreadsheet, in the designated space.
2. Obtain averages for the values in the two sets of controls:
   (a) Positive control—wells in column 1 rows C-F (sample bacteria processed without any antibiotic).
   (b) Background-wells in column 1 rows G and H (sample bacteria grown without adding Buffer B, but still processed with all other steps). This represents non-labeled bacteria.
3. Determine the Relative Response Ratio (RRR) for each test well as follows:
   (a) Subtract the value of each test well from the positive control average (positive control average-value of test well).
   (b) Subtract the background value from the positive control average (positive control average-background value)
   (c) Divide the two values as follows below in Formula (1):

$$Relative Response Ratio = \frac{Average Positive Control Value - Average Sample Value}{Average Positive Control Value - Background Value}$$

(d) Store/Save the Relative Response Ratios in an array representing a map of the values, normalized for the relative labeling efficiency of the bacteria being tested.
4. Use a logic statement, if/then to convert the data from the array in 15 to a “Truth Table” using the supplied cut off values (calibrated against the specific standard being used in the assay) as follows: For each test well, if the RRR value from the table is higher than the cut off, then the logic statement returns a value of zero, if the number is lower than the cut off, then the value of 1 is returned. Any small percentage of failed wells with anonymously high or low values should be scored according to the values of adjacent wells based on a set of rules. If a well is scored zero, it is a valid result if the next higher concentration well is also scored as zero.
5. Determine the MICs as follows. For each 96-well plate processed there is a map of the corresponding antibiotics and their concentrations at each well location. The zero in the table corresponds to wells where the concentration is effective in inhibiting the bacterial metabolism to a level below the predetermined threshold value. A simple function to look up the lowest concentration of each antibiotic that reaches the threshold value (a zero in the table, in this example) identifies the MIC for that antibiotic. In many cases, the bacteria may be able to grow at all concentrations of a particular antibiotic tested, in which case the software (all the values in the column are “1”), returns the value “resistant to greater than the top value tested.” Conversely, if the bacteria are sensitive to all the concentrations tested then the value is sensitive to less than the lowest value tested. The results are then used to populate a report that includes the MIC of each antibiotic tested for a particular sample.

For each test, Quality Control and clinical specimen results are interpreted as positive or negative based on the difference between treated and untreated samples. If either control does not meet acceptance criteria, results are considered invalid, and specimens must be re-tested. If the sterility control wells (column 1, rows A and B) show bacterial growth, then the entire plate is a failed test and must be discarded. All assay controls should be examined prior to the interpretation of results. If results of assay controls are not valid, results of specimens cannot be interpreted, and the assay must be repeated.

### EXPERIMENTAL RESULTS

### Testing Fluorogenic and Fluorescent Dyes

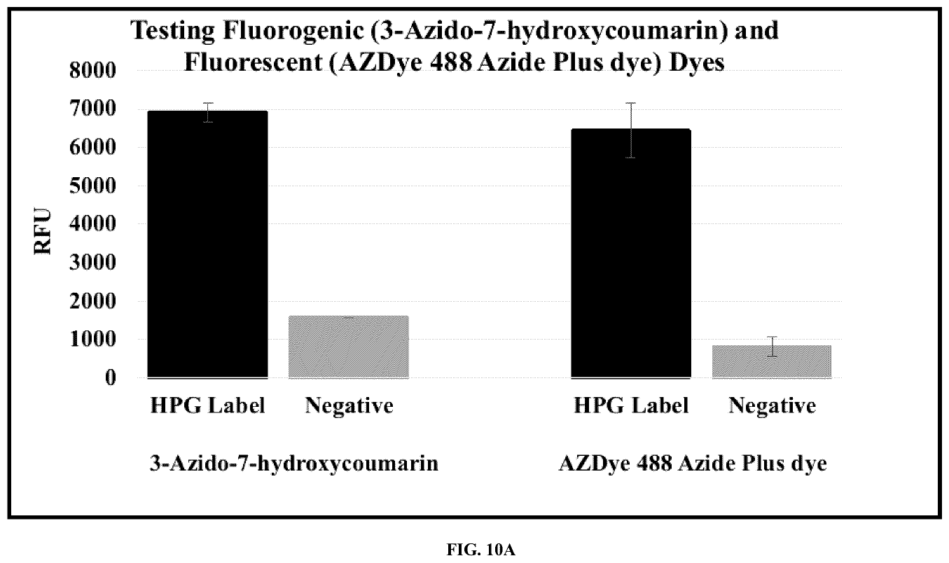

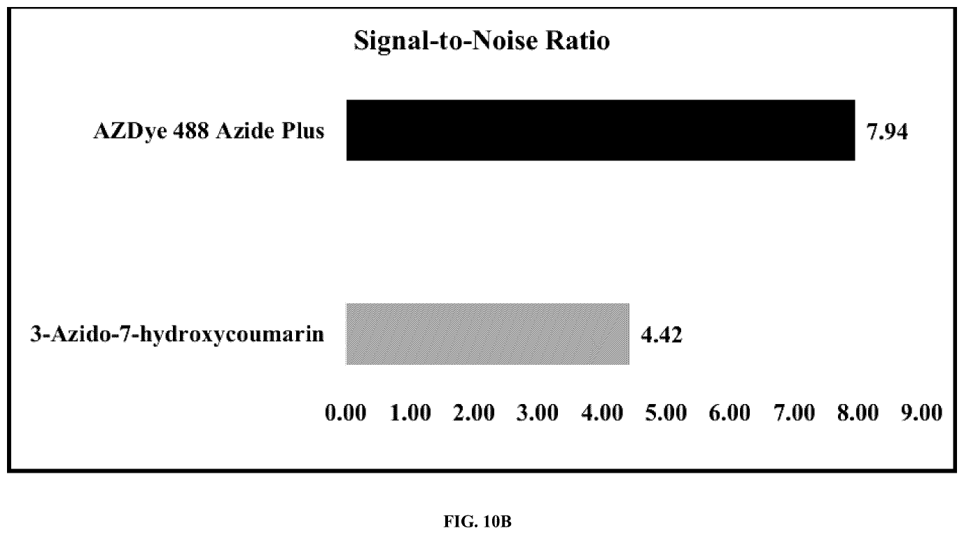

The following dyes (one fluorogenic and one fluorescent) were used to label an E. coli strain (ATCC strain 25922) using the CytoSPAR BLAST system: (i) 3-Azido-7-hydroxycoumarin dye-Fluorogenic dye (Jena Biosciences, cat #CLK-FA047-1); and AZDye 488 Azide Plus dye-Fluorescent dye (Click Chemistry Tools, #1475-25). Samples were processed and incubated with the same labeling buffer. The negative control had no labeling buffer. Each sample was then incubated with its own respective tagging buffer as follows (a) 3-Azido-7-hydroxycoumarin dye; and (ii) AZDye 488 Azide Plus dye. The results shown in FIG. 10 indicate that the system was able to label bacteria using both fluorogenic and fluorescent dyes. Using the fluorescent dye (AZDye 488 Azide Plus), the background (negative control) signal was greatly reduced by washing thereby resulting in a greater signal-to-noise ratio. Both dyes gave acceptable signal-to-noise ratios and can be used in the disclosed system. In addition, the fluorogenic dye (3-Azido-7-hydroxycoumarin) can be used for tagging without the need for a washing step.

### Antibiotic Susceptibility Test

The following 4 strains of bacteria were tested against varying concentrations of nitrofurantoin using the CytoSPAR BLAST system at 1.5×10⁸ CFUs/mL:
1. Gram-negative strains:
   a) E. coli strain #25922 (ATCC)-Susceptible to nitrofurantoin.
   b) E. coli strain #551 (CDC)-Resistant to nitrofurantoin.
   c) E. coli strain #14 (CDC)-Intermediate resistance to nitrofurantoin.
2. Gram-positive strain:
   a) S. saprophyticus strain #15305 (ATCC).
3. Controls:
   a) Positive control:
      No antibiotics.
      Cells were incubated with labeling buffer.
   b) Negative control:
      No antibiotics.
      Cells were incubated without labeling buffer.

The following concentrations of nitrofurantoin were tested against each bacterial strain: 0.5 μg/mL, 1 μg/mL, 2 μg/mL, 4 μg/mL, 8 μg/mL, 16 μg/mL, 32 μg/mL, 64 μg/mL, 128 μg/mL, 256 μg/mL, 512 μg/mL, and 1,024 μg/mL. These twelve concentrations include the antibiotic breakpoint based on the FDA recommended breakpoints for nitrofurantoin (see TABLE 4, below), six concentrations below the breakpoint and five concentrations above the breakpoint in two-fold dilution.

TABLE 4
Nitrofurantoin breakpoints for E. coli and
S. saprophyticus as reported by the FDA and CLSI.

| | | Min | Max | No. | | | | | | |
|---|---|---|---|---|---|---|---|---|---|---|
| | | Conc. | Conc. | of | CLSI (μg/mL) | | | FDA (μg/mL) | | |
| Bacteria | Drugs | (μg/mL) | (μg/mL) | Conc. | S | I | R | S | I | R |
| E. coli | Nitrofurantoin | 4 | 128 | 6 | 32 | 64 | 128 | 32 | 64 | 128 |
| S. saprophyticus | Nitrofurantoin | 8 | 128 | 5 | 32 | 64 | 128 | 32 | 64 | 128 |

TABLE 5
Nitrofurantoin concentrations showing the breakpoint and MIC range
for E. coli, S. saprophyticus and S. aureus as reported by the
FDA and CLSI. The dark grey highlight represents the CLSI QC
range (4-16 μg/mL) while the light grey highlight
represents the breakpoint (32 μg/mL).
Nitro (μg/mL)

2
4
8
16
32
64
128
256

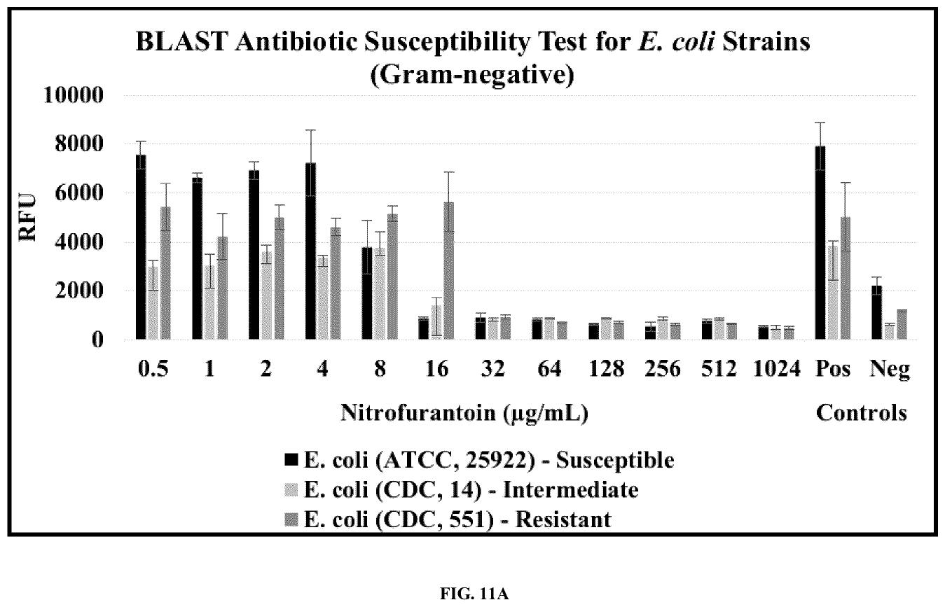

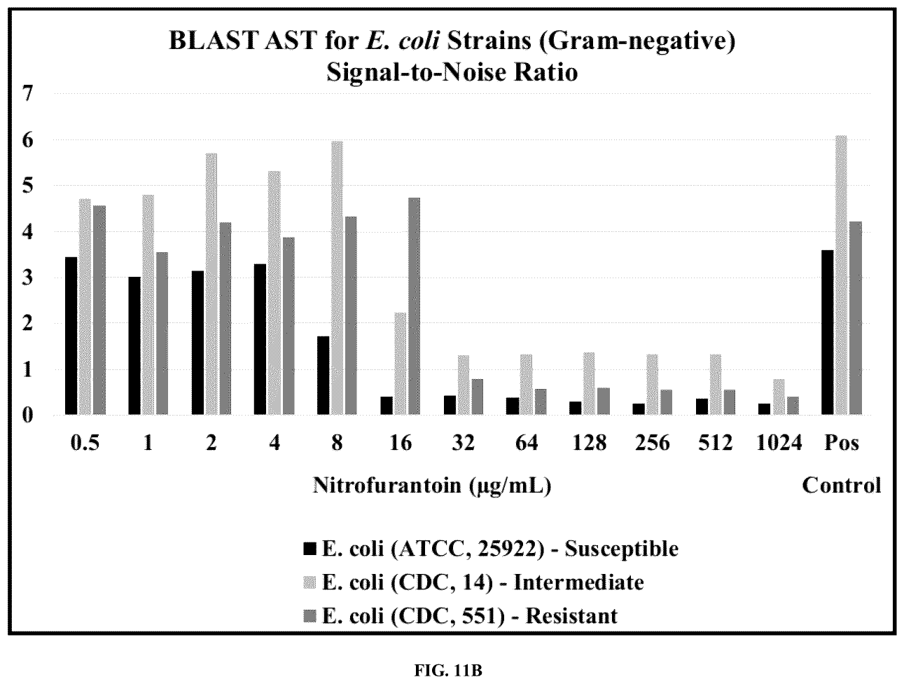

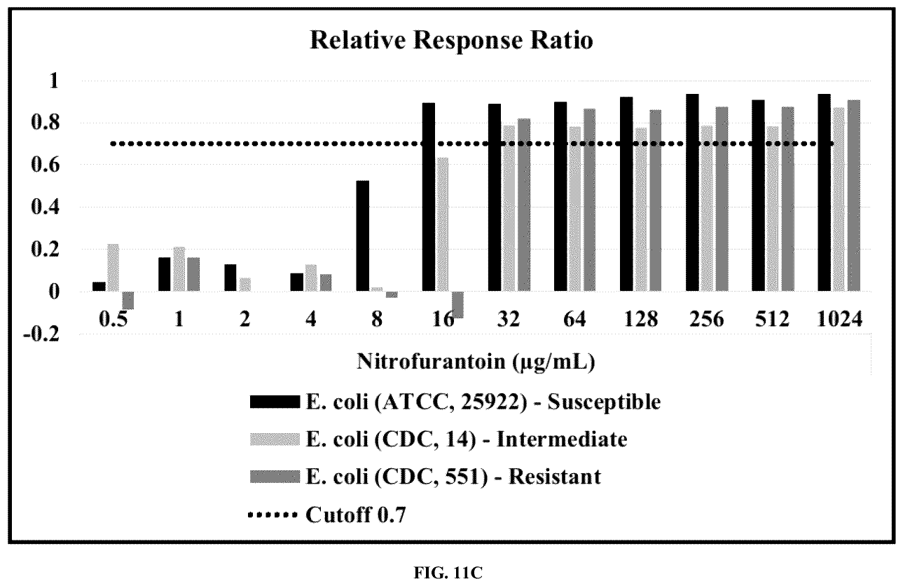

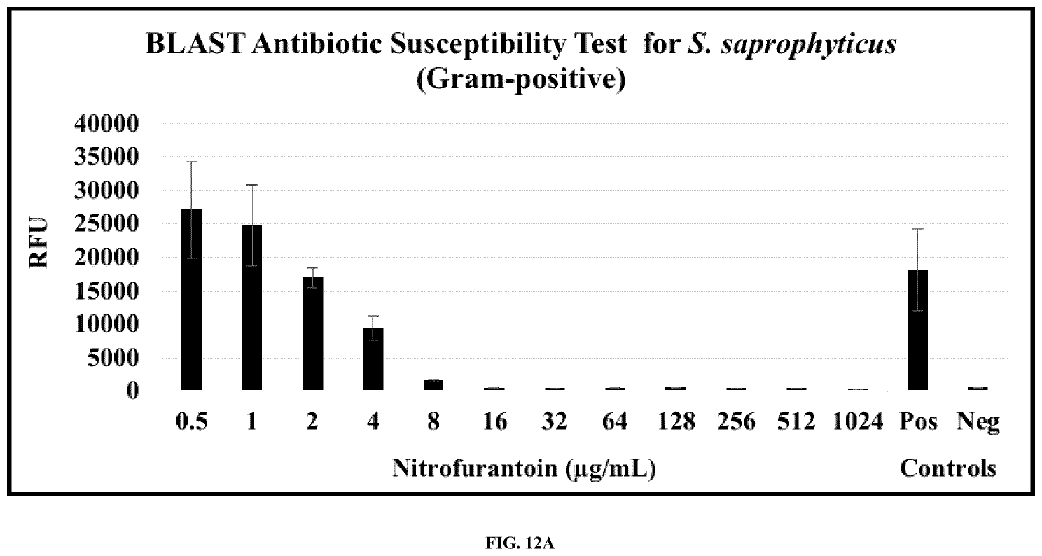

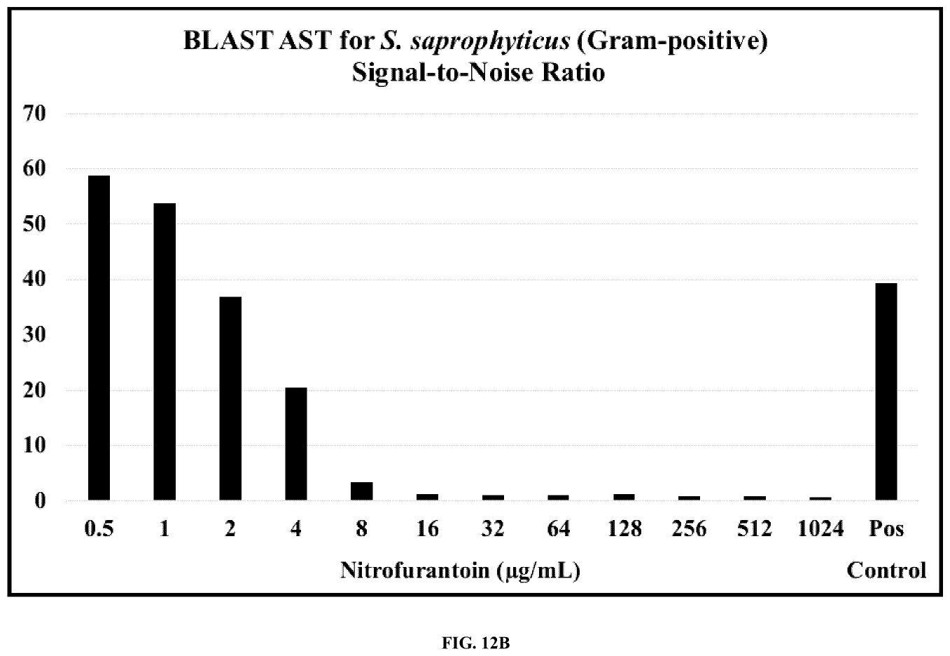

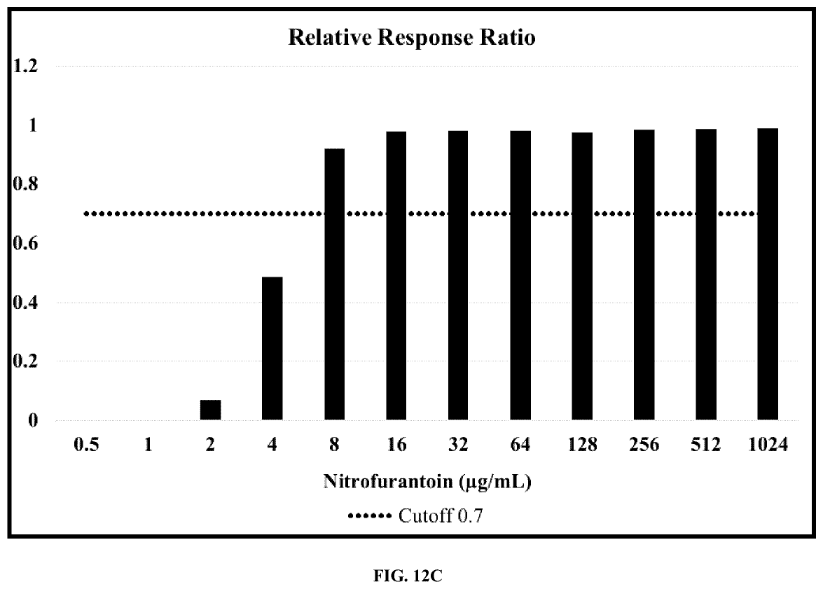

The results shown in FIGS. 11 and 12 indicate that the CytoSPAR BLAST system was successfully used to perform antibiotic susceptibility test on four strains of bacteria: three E. coli (Gram-negative) strains and one S. saprophyticus (Gram-positive) strain. The three E. coli strains comprised of one susceptible, one intermediate and one resistant strain to nitrofurantoin. Using the disclosed BLAST system, a table of the relative response ratio was generated (see FIGS. 11C and 12C) and MIC values are determined using the supplied pre-determined cut-off value (0.7 in this case). The concentration at which the RRR crosses the cutoff line represents the BLAST MIC. Results presented in FIG. 11C show the nitrofurantoin BLAST MIC for E. coli strains as; 16 μg/mL (susceptible strain), 32 μg/mL (intermediate strain), and 32 μg/mL (resistant strain). The nitrofurantoin BLAST MIC for S. saprophyticus is 8 μg/mL as represented in FIG. 12C. These results are comparable to the CLSI QC range of 4-16 μg/mL (see TABLE 5, above).

### Assay Sensitivity

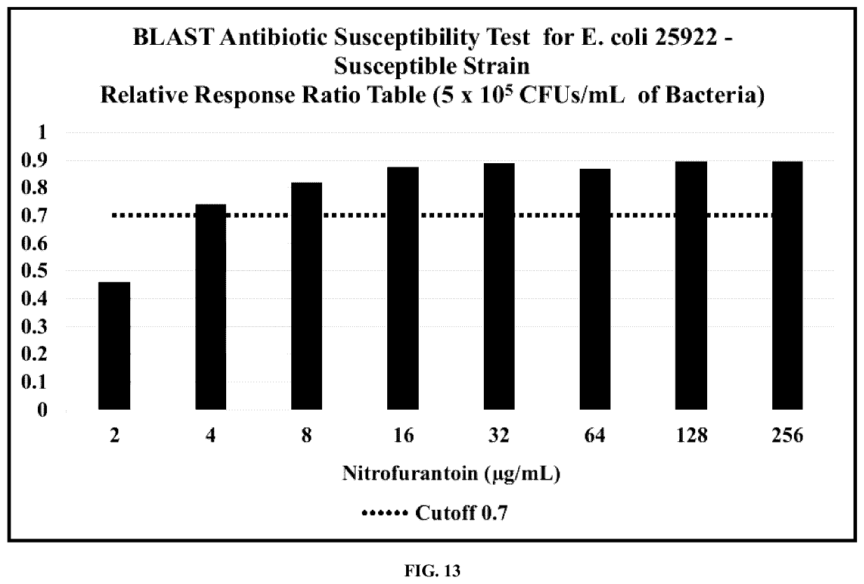

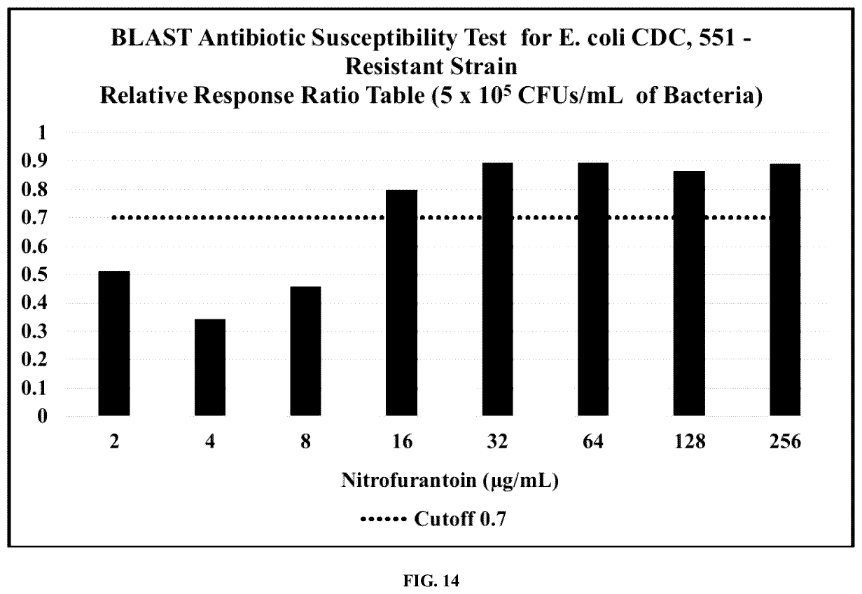

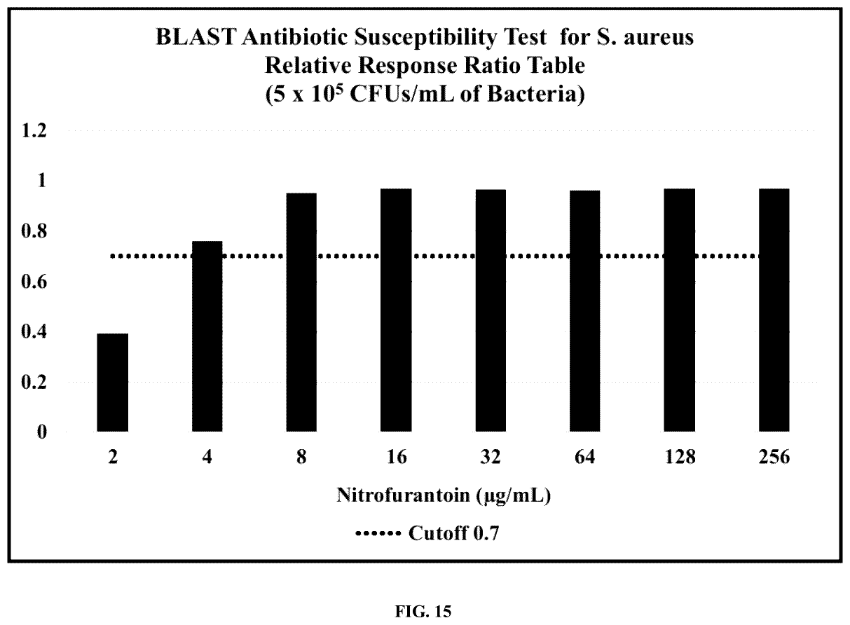

The nitrofurantoin results described in FIGS. 11 and 12 were generated by testing at 1.5×10⁸ CFUs/mL of bacteria. However, lower concentrations of bacteria were also tested using 5×10⁵ CFUs/mL and the results described in FIG. 13 show that similar BLAST MIC values were attained for the E. coli susceptible strain (ATCC strain #25922). Comparable results were also achieved when the E. coli resistant strain (CDC #551) and S. aureus (ATCC #29213) were tested against nitrofurantoin (see FIGS. 14 and 15 respectively). These results show that the system is highly sensitive and can work with very low concentrations of bacteria in the sample. All the BLAST MICs are comparable to the CLSI QC range of 4-16 μg/mL, whether the assay is performed using an inoculum of 1.5×10⁸ CFUs/mL or 5×10⁵ CFUs/mL.

### BLAST System Optimization

In one implementation, the BLAST system was optimized to reduce the total time of the assay to 5 hours and 5 minutes. This was achieved by reducing the acclimatization and labeling time, together with elimination of two wash steps. The sensitivity of the assay was also improved 300-fold from the original inoculum of 1.5×10⁸ CFU/mL to 5×10⁵ CFU/mL. In addition, the BLAST kit (see FIG. 3) was designed and manufactured by consolidating all assay reagents into 5 buffers. This was achieved by combining various components of the tagging buffer into just two buffers. The short-term stability of the tagging buffer components and the complete tagging buffer mix was tested by storing the buffers at different conditions for a certain period of time. The long-term stability of the kit is greatly enhanced by lyophilization of Buffer C1 and Buffer C2 into lyobeads so that the entire kit can be shipped under ambient conditions and stored at room temperature for long time periods (e.g., longer than a year). This eliminates the need for cold storage. Alternate dyes have been successfully tested and may be used in the BLAST assay as an alternative to previously used and tested dyes.

### BLAST Kit Design

One implementation of the BLAST kit contains five buffers: Buffer A for the acclimatization step, Buffer B for the labeling step, Buffers C1 and C2 for the tagging step, and Buffer W for the washing step in the BLAST assay. In addition, the kit contains a 2 L bottle for wash buffer dilution. Another implementation contains all the listed buffers plus filter plates, plate seals, and antibiotics for the assay. Each kit has enough materials to test 48 samples against 11 antibiotics. One filter plate is sufficient for testing one sample against 11 antibiotics.

Bacterial Strains and Antibiotics that have been Tested by BLAST

Using the optimized BLAST assay conditions described herein, an antibiotic susceptibility test was performed for E. coli (ATCC strain #25922) and S. aureus (ATCC strain #29213) against three antibiotics, cefazolin, doxycycline, and levofloxacin using either 5×106 CFUs/mL, or 5×10⁵ CFUs/mL of bacteria. The varying concentrations of each antibiotic were tested in accordance with the disclosed BLAST assay protocol. In each case, the fluorescent signal of bacterial response to varying concentrations of the antibiotic was transformed into the Relative Response Ratio (RRR) to determine the relative labeling efficiency of the bacteria as described herein. Briefly, the value of each test well is subtracted from the value of the positive control average and then divided by the value of the positive control average minus the background. Numbers close to zero indicate very little inhibition by the antibiotic and numbers close to one indicate a significant degree of inhibition. A cutoff value of 0.8 is set in the BLAST assay system. The antibiotic concentration at which the RRR crosses the cutoff value represents the MIC.

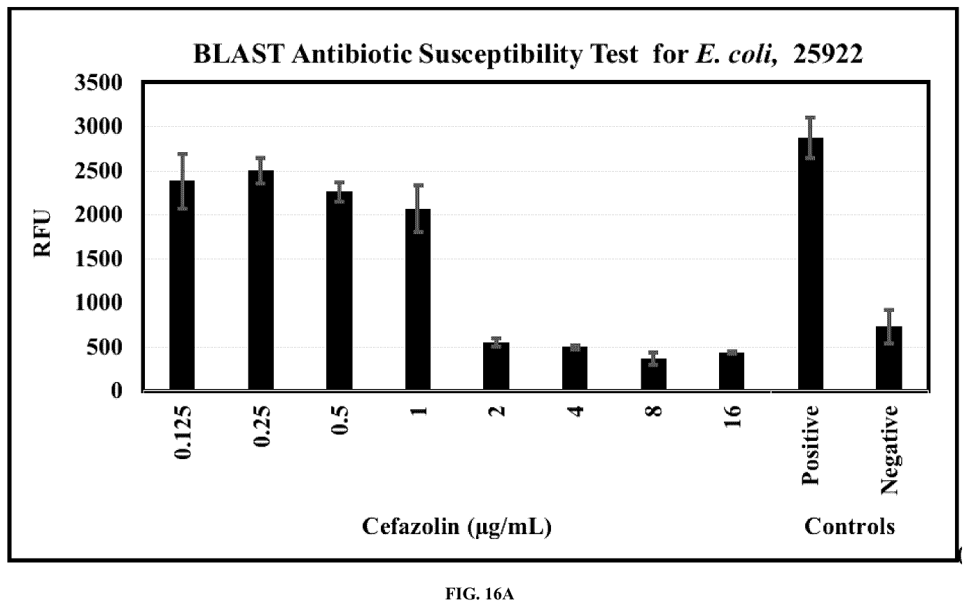

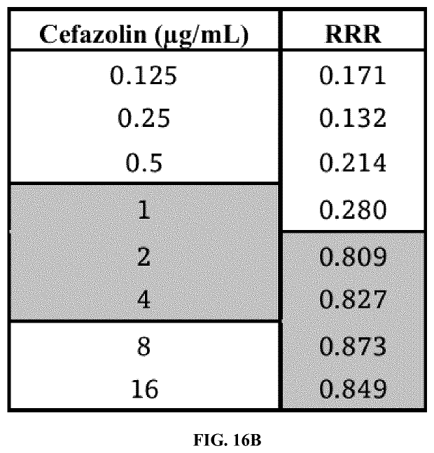

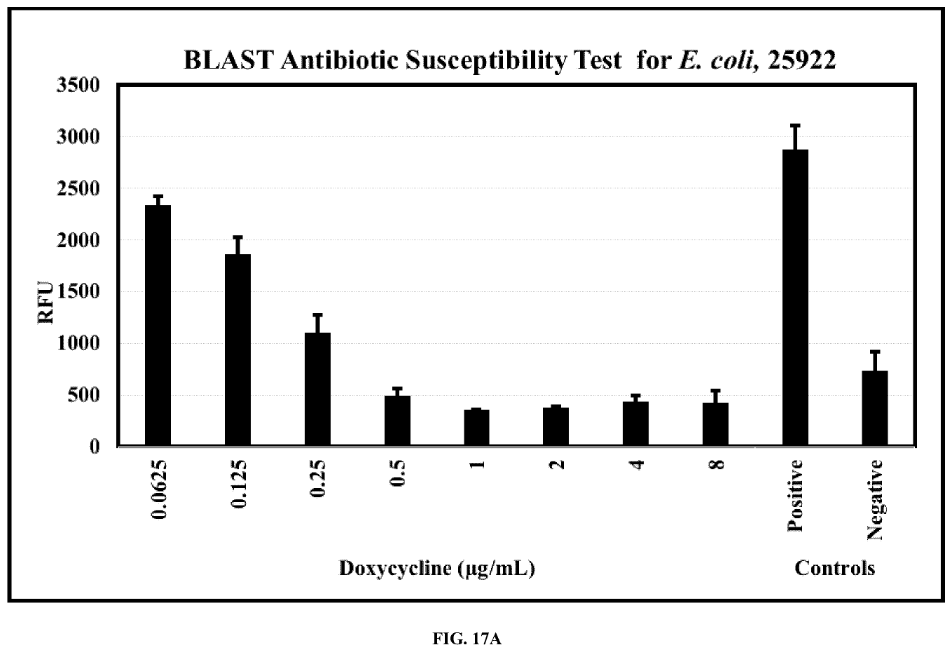

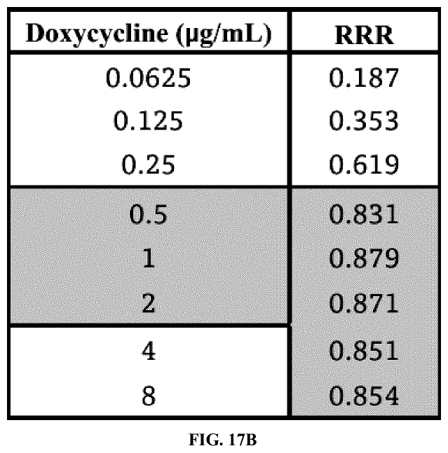

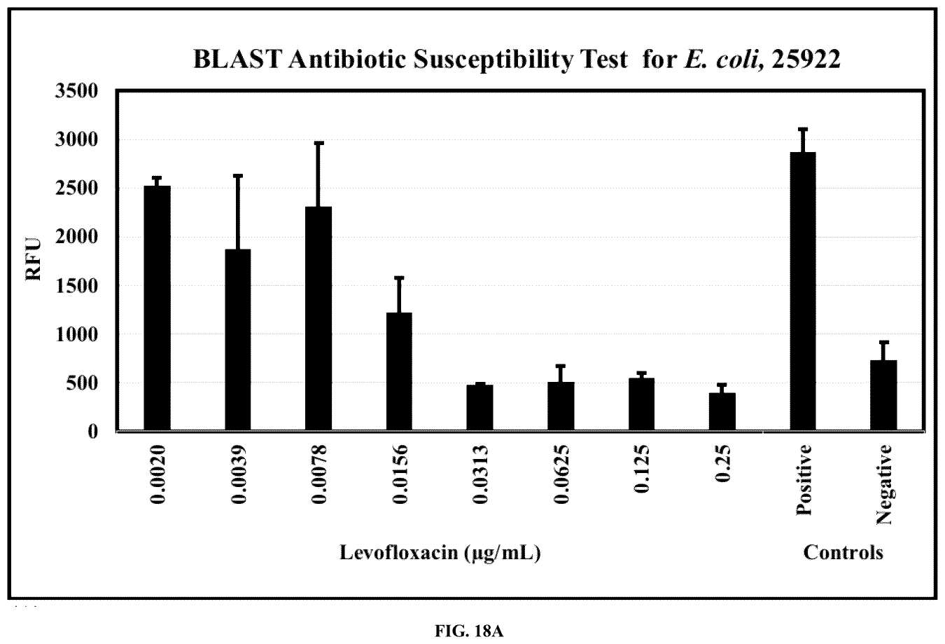

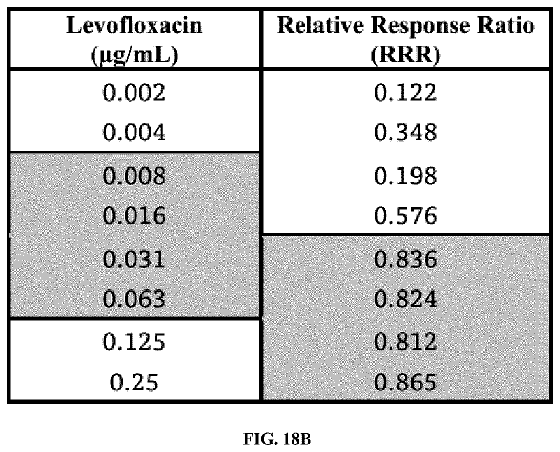

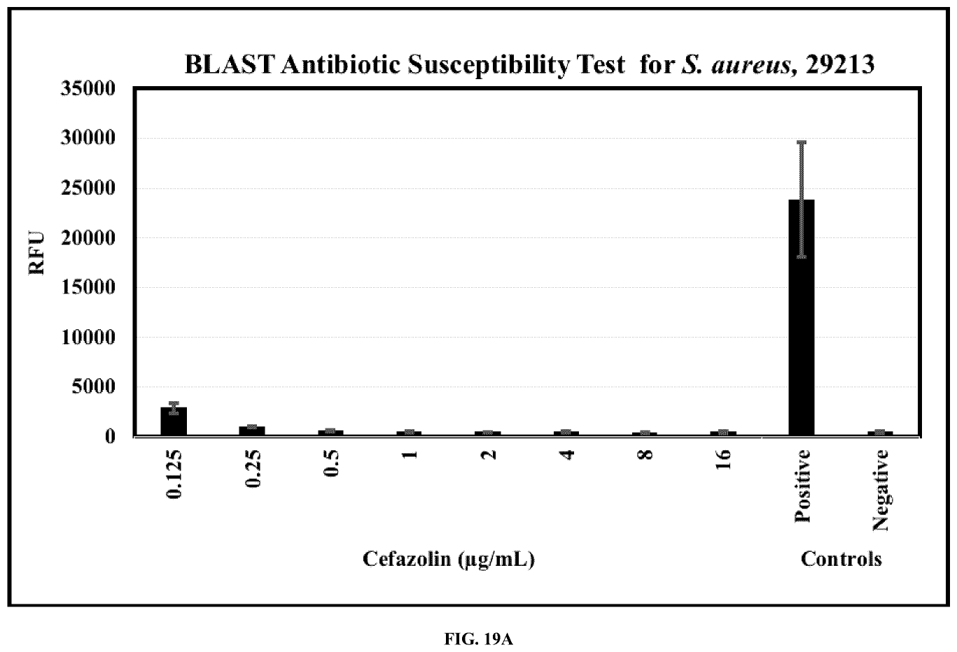

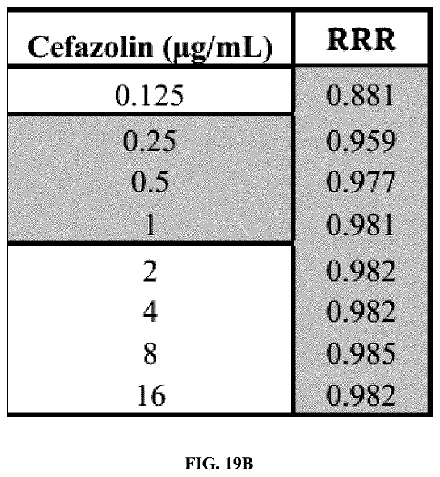

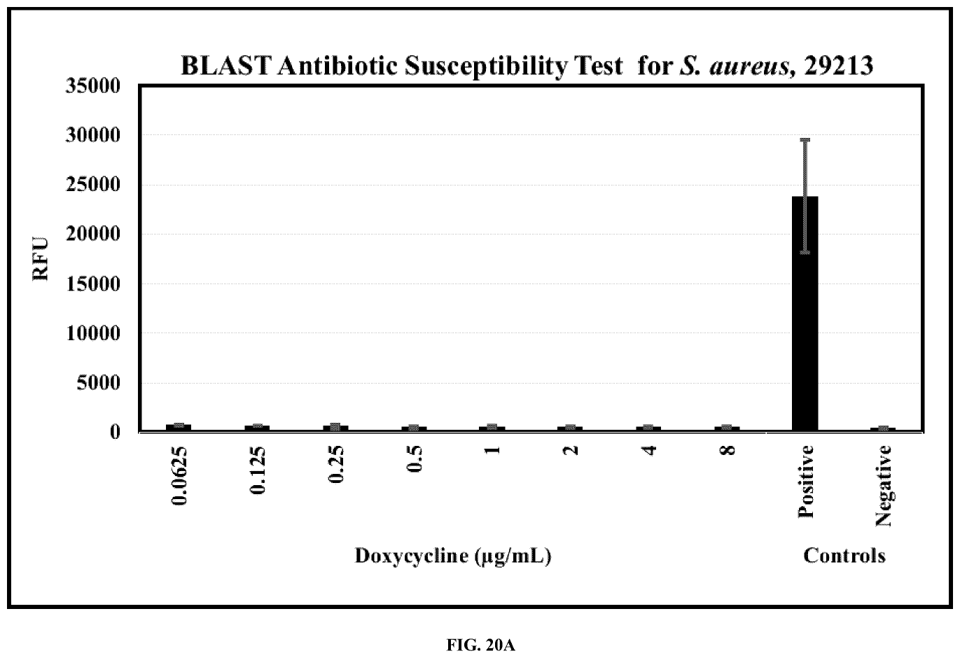

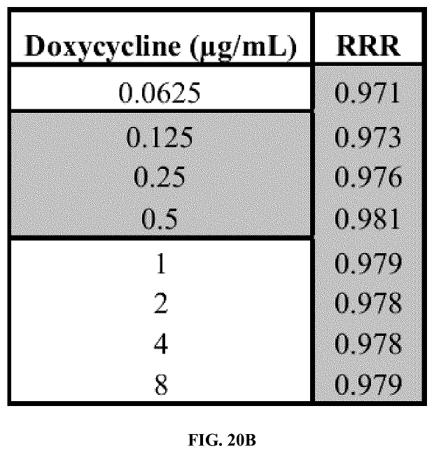

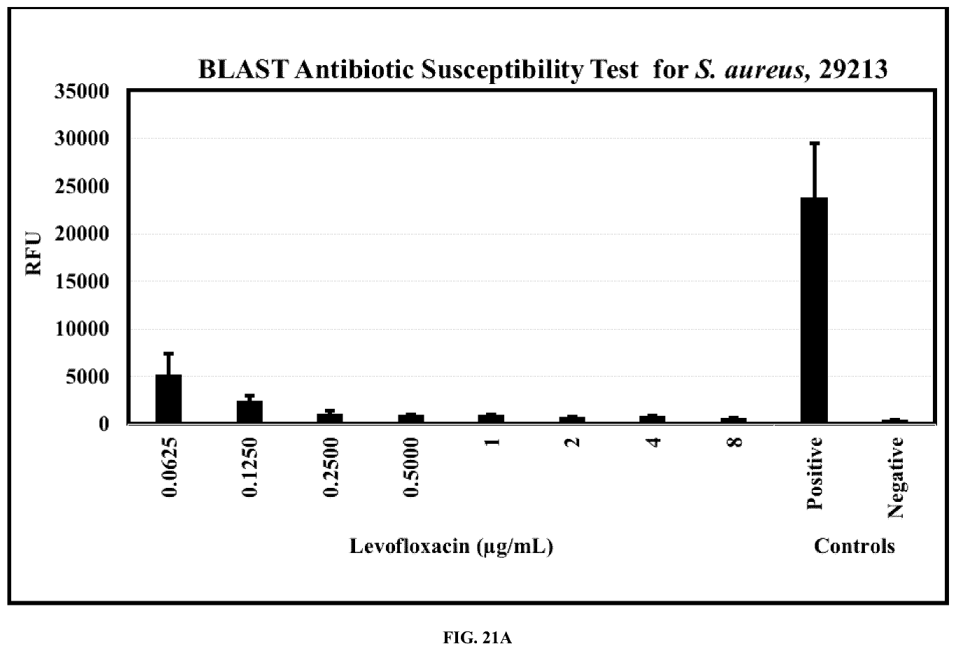

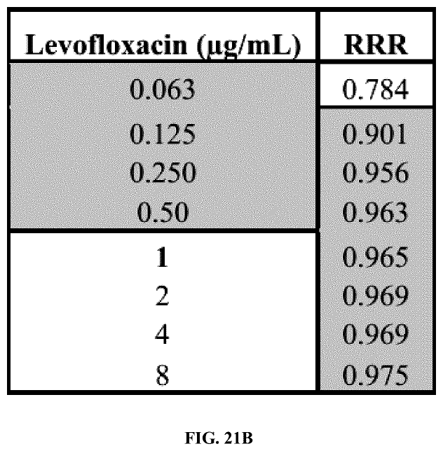

Based on the BLAST results shown in FIGS. 16-21 for testing E. coli (ATCC strain #25922) and S. aureus (ATCC strain #29213) against cefazolin, doxycycline, and levofloxacin, BLAST MICs for the three tested antibiotics were within the CLSI/FDA QC range. In two instances, the BLAST MIC values for S. aureus were lower than the lowest tested antibiotic concentration. Testing E. coli against cefazolin (see FIG. 16) gave a BLAST MIC of 2 μg/mL which is comparable to the CLSI/FDA QC range of 1-4 μg/mL. Similarly, when E. coli was tested against doxycycline, it resulted in a BLAST MIC value of 0.5 μg/mL (see FIG. 17). This value falls within the CLSI/FDA QC range of 0.5-2 μg/mL. A similar trend was reported for E. coli against levofloxacin (see FIG. 18), and S. aureus against levofloxacin (see FIG. 21). When S. aureus was tested against cefazolin (see FIG. 19) and doxycycline (see FIG. 20), the resultant BLAST MIC values of 0.125 g/mL and 0.0625 μg/mL were reported respectively. The two BLAST MICs were lower than the lowest concentrations of the antibiotics tested in each case.

Following optimization of the BLAST assay, two other tests were performed in which 12 antibiotics (covering different classes) and 8 bacterial strains (Gram-positive and Gram-negative) were assayed using the same method as described above. Different bacterial strains were tested against different antibiotics and for each pair tested, the BLAST MIC value was compared to the CLSI QC range where available, or to the reference broth microdilution (rBMD) MIC which was determined in parallel with the BLAST assay following the CLSI protocols (see references [8] [9]). Results were computed and are summarized in TABLE 6 and TABLE 7, below.

In one of the assays (see TABLE 6, below), two quality control strains (E. coli ATCC 25922, and S. aureus ATCC 29213) were each tested against 12 antibiotics (nitrofurantoin, meropenem, ceftriaxone, ciprofloxacin, cephalexin, cefuroxime, augmentin, ampicillin, cefoxitin, doxycycline, levofloxacin, and cefazolin) comprising different classes of antibiotics. In the second study (see TABLE 6), seven bacterial strains (E. coli ATCC 25922, E. coli LSI 770, S. aureus ATCC 29213, K. pneumoniae LSI 4552, K. pneumoniae LSI CT1045, P. mirabilis LSI 4698, and P. mirabilis LSI 4933) were each tested against doxycycline, levofloxacin, and cefazolin using both BLAST and the rBMD. Each BLAST MIC value was compared to the corresponding rBMD MIC and results (see FIG. 6) show that most of the BLAST MICs were similar to, or within 1-2 dilutions of the reference broth microdilution MICs. Only two out of the seven strains were QC strains, therefore most BLAST MICs were not compared to the CLSI QC range. Two other strains, S. saprophyticus and S. pyogenes were successfully tested but the results were not included in the table.

TABLE 6
BLAST MIC results compared to the reference broth
microdilution MICs and CLSI quality control range.
S, I, R = susceptible, intermediate, resistant.

| | | | MIC (μg/mL) | |
|---|---|---|---|---|
| | | | | CLSI QC |
| Bacteria Strain | Antibiotic | S, I, R | BLAST | Range¹ |
| E. coli | Nitrofurantoin | Susceptible | 4 | 4.0-16.0 |
| ATCC 25922 | Meropenem | Susceptible | 0.0156 | 0.008-0.06 |
| | Ceftriaxone | Susceptible | 0.0625 | 0.03-0.12 |
| | Ciprofloxacin | Susceptible | 0.0156 | 0.004-0.016 |
| | Cephalexin | Susceptible | 16 | 1.0-4.0 |
| | Cefuroxime | Susceptible | 4 | 2.0-8.0 |
| | Augmentin | Susceptible | 2 | 2/1-8/4 |
| | Ampicillin | Susceptible | 4 | 2.0-8.0 |
| | Cefoxitin | Susceptible | 4 | 2.0-8.0 |
| | Doxycycline | Susceptible | 0.5 | 0.5-2.0 |
| | Levofloxacin | Susceptible | 0.031 | 0.008-0.06 |
| | Cefazolin | Susceptible | 2 | 1.0-4.0 |
| S. aureus | Nitrofurantoin | Susceptible | 4 | 8.0-32.0 |
| ATCC 29213 | Meropenem | Susceptible | 0.0625 | 0.03-0.12 |
| | Ceftriaxone | Susceptible | 1 | 1.0-8.0 |
| | Ciprofloxacin | Susceptible | 0.25 | 0.12-0.5 |
| | Cephalexin | Susceptible | 1 | 0.25-1 |
| | | | | (Cefazolin) |
| | Cefuroxime | Susceptible | 0.25 | 0.5-2 |
| | Augmentin | Susceptible | 0.125 | Not available |
| | Ampicillin | Susceptible | 0.50 | 0.5-2 |
| | Cefoxitin | Susceptible | 1 | 1.0-4.0 |
| | Doxycycline | Susceptible | 0.0156 | 0.12-0.5 |
| | Levofloxacin | Susceptible | 0.25 | 0.06-0.5 |
| | Cefazolin | Susceptible | 0.125 | 0.25-1.0 |
| P. aeruginosa | Nitrofurantoin | Resistant | >256 | Not available |
| ATCC 27853 | Levofloxacin | Susceptible | 8 | 0.5-4.0 |

¹CLSI M100-Ed34E

TABLE 7
BLAST MIC results compared to the reference broth microdilution MICs and CLSI
quality control range. S, I, R = susceptible, intermediate, resistant.

| | | | MIC (μg/mL) | | | Dilution |
|---|---|---|---|---|---|---|
| Bacteria | | | | | CLSI QC | Difference |
| Strain | Antibiotic | S, I, R | BLAST | rBMD¹ | Range² | (BLAST-rBMD) |
| E. coli | Doxycycline | Susceptible | 0.14 | 0.5 | 0.5-2.0 | −2 |
| ATCC 25922 | Levofloxacin | Susceptible | 0.01 | 0.02 | 0.008-0.06 | −1 |
| | Cefazolin | Susceptible | 1 | 2 | 1.0-4.0 | −1 |
| E. coli | Doxycycline | Resistant | 71 | 64 | NA | 0 |
| LSI 770 | Levofloxacin | Resistant | 36 | 32 | NA | 0 |
| | Cefazolin | Resistant | >142 | >256 | NA | both off-scale |
| S. aureus | Doxycycline | Susceptible | 0.02 | 0.12 | 0.12-0.5 | −2 |
| ATCC 29213 | Levofloxacin | Susceptible | 0.28 | 0.25 | 0.06-0.5 | 0 |
| | Cefazolin | Susceptible | 0.14 | 0.5 | 0.25-1.0 | −2 |
| K. pneumoniae | Doxycycline | Susceptible | 1 | 2 | NA | −1 |
| LSI 4552 | Levofloxacin | Susceptible | 0.06 | 0.12 | NA | −1 |
| | Cefazolin | Susceptible | 2 | 8 | NA | −2 |
| K. pneumoniae | Doxycycline | Resistant | | 8 | NA | |
| LSI CT1045 | Levofloxacin | Resistant | >71 | 128 | NA | |
| | Cefazolin | Resistant | >142 | >256 | NA | both off-scale |
| P. mirabilis | Doxycycline | Susceptible | 4.4 | 32 | NA | −3 |
| LSI 4698 | Levofloxacin | Susceptible | 8.9 | 16 | NA | −1 |
| | Cefazolin | Susceptible | 4.4 | 16 | NA | −2 |
| P. mirabilis | Doxycycline | Susceptible | 1.1 | 4 | NA | −2 |
| LSI 4933 | Levofloxacin | Susceptible | 1.1 | 1 | NA | 0 |
| | Cefazolin | Susceptible | 4.4 | 8 | NA | −1 |

¹Geometric Mean MIC (rounded to nearest 2-fold dilution; n = 3)
²CLSI M100-Ed34E
NA = Not Applicable

In summary, the BLAST method was optimized to generate MIC values that fall within the CLSI QC ranges for the tested antibiotics and bacterial strains. The test results were achieved in less than 6 hours without compromising the sensitivity and reproducibility of the system.

### Spiked Urine Test

Normal pooled urine was inoculated with cultures of either E. coli ATCC 25922, S. aureus ATCC 29213, or E. coli LSI 770 from blood agar plates. The inoculated urine cultures were incubated overnight at 35+2° C. Each urine culture was centrifuged, and the sediment used to prepare the BLAST inoculum of 5.0×10⁵ CFU/mL to start the assay as described in the protocols section. Samples were tested against different concentrations of doxycycline, levofloxacin, and cefazolin in a BLAST assay. The spiked urine samples were tested in parallel with isolates and the resultant BLAST MIC values were compared. In addition to the BLAST test, all the samples were also tested using the reference broth microdilution (rBMD) method and the MIC values were compared.

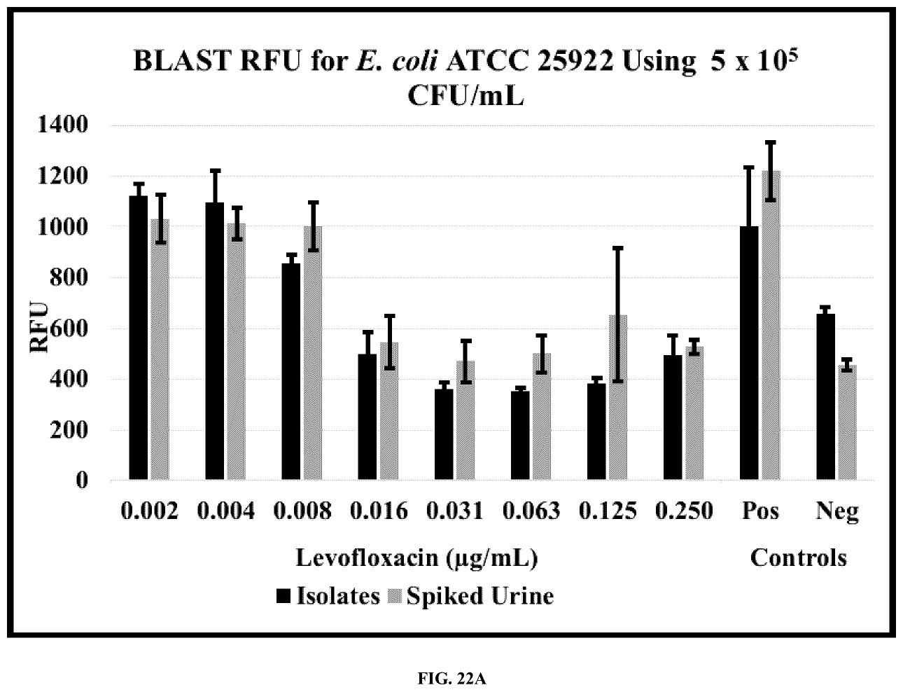

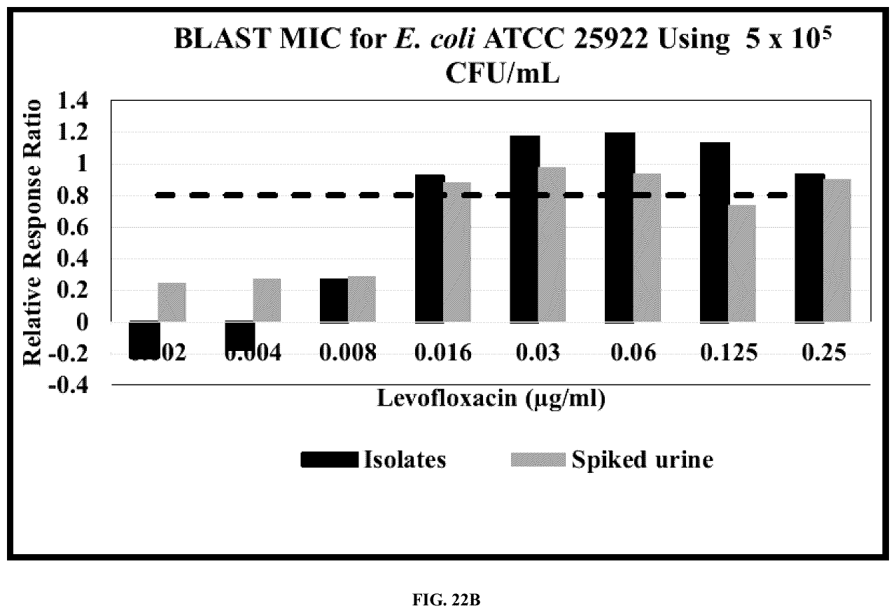

The results provided in FIG. 22 show the response of E. coli ATCC 25922 against different concentrations of levofloxacin using the BLAST assay. Results show that the spiked urine sample and isolate gave the same BLAST MIC of 0.016 μg/mL which is within the CLSI QC range of 0.008-0.063 μg/mL for levofloxacin against E. coli ATCC 25922. Other results presented in TABLE 8, below, show that each sample gave a similar BLAST MIC when tested as a spiked urine sample or isolate. Similarly, each BLAST MIC was within the corresponding CLSI QC range and comparable to the corresponding rBMD MIC. In summary, the BLAST assay was successfully used for AST using spiked urine samples to emulate direct sample testing.

TABLE 8
Using BLAST to determine MIC values for spiked urine samples and isolates of
E. coli ATCC 25922, S. aureus ATCC 29213, and E. coli LSI 770 against doxycycline,
levofloxacin, and cefazolin at 5 × 10⁵ CFU/mL. Each MIC was compared
to the corresponding CLSI QC range and/or corresponding rBMD MIC.

| | | | | | | | Dilution | |
|---|---|---|---|---|---|---|---|---|
| | | | MIC (μg/mL) | | | | Difference | |
| | | | BLAST | | | | (BLAST-rBMD) | |
| Bacteria | | | Spiked | | | CLSI QC | Spiked | |
| Strain | Antibiotic | S, I, R | Urine | Isolates | rBMD¹ | Range² | Urine | Isolates |
| E. coli | Doxycycline | Susceptible | 0.28 | 0.14 | 0.5 | 0.5-2.0 | −1 | −2 |
| ATCC | Levofloxacin | Susceptible | 0.01 | 0.01 | 0.016 | 0.008-0.06 | −1 | −1 |
| 25922 | Cefazolin | Susceptible | 1.11 | 4.44 | 2 | 1.0-4.0 | −1 | 1 |
| S. aureus | Doxycycline | Susceptible | 0.02 | 0.02 | 0.12 | 0.12-0.5 | −2 | −2 |
| ATCC | Levofloxacin | Susceptible | 0.28 | 0.28 | 0.25 | 0.06-0.5 | 0 | 0 |
| 29213 | Cefazolin | Susceptible | 0.28 | 0.14 | 0.5 | 0.25-1.0 | −1 | −2 |
| E. coli | Doxycycline | Resistant | 71 | 71 | 64 | NA | 0 | 0 |
| LSI 770 | Levofloxacin | Resistant | 35.6 | 35.6 | 32 | NA | 0 | 0 |

| | | | | | | |
|---|---|---|---|---|---|---|
| Cefazolin | Resistant | >142 | >142 | >256 | NA | both off-scale |

¹Geometric Mean MIC (rounded to nearest 2-fold dilution; n = 3)
²CLSI M100-Ed34E
NA = Not Applicable

### Alternative Reducing Agents: Aldose Reduction Catalysis

As will be appreciated by one skilled in the art, the use of click chemistry reactions using copper catalysis in tagging bacteria for use in a diagnostic kit may create challenges regarding the creation of shelf-stable liquid reagents. Ascorbic acid, which is commonly used in laboratory experiments, may exhibit reduced stability as a liquid and may be incompatible with other click chemistry reaction buffer components. Solutions containing ascorbic acid may become unstable when assembled and must be used immediately. Such instability may be due to the formation of oxygen free radicals associated with the strong reducing nature of ascorbic acid. Preparation of the click chemistry buffer (Sherratt et al., 2017) is a time-consuming series of steps that increases the risk of error in the assay. Additionally, manufacturing and accounting for separate reagents is not cost effective. Accordingly, a suitable reducing agent that is milder, more shelf-stable, and that is compatible with the disclosed BLAST assay is desirable.

In contrast with aqueous ascorbic acid, solutions of certain aldose sugars are much more stable and are milder reducing agents. The pH of the solution during copper ion reduction should also be compatible with the other components of the click chemistry reaction, for example, it should not damage the azide dye or precipitate copper I in the presence of the ligand. Disclosed herein are sugars having the proper structure for reducing copper under the reaction conditions used in the disclosed BLAST assay. Glyceraldehyde, glucose, ribose, and closely related aldose sugars are compatible with the formulations disclosed herein. The use of these sugars is non-toxic and cost-effective, and can be used to create shelf-stable liquids that can be shipped at room temperature as a kit component.

Experiments were conducted to compare the BLAST assay response using sodium ascorbate as the reductant with assay responses using four different sugars: glyceraldehyde, glucose, ribose, and sucrose. Glucose has been shown to reduce copper (II) to copper (I) at elevated pH values (Singh et al., 1970) and ribose has been shown to have a greater percentage of acyclic aldehyde compared with glucose (Zhu et al., 2001 and Drew et al., 1998). Although both glucose and ribose are predominantly in the cyclic hemiacetal form, the acyclic aldehyde is required for the formation of the ene-diol, the intermediate shown to be responsible for the reduction of Cu (II) to Cu(I) by aldoses (Singh et al., 1970). Glyceraldehyde was also tested given its natural acyclic form. Sucrose, a non-reducing sugar, was used as the negative control. Ascorbate, which is the positive control, is predominantly in the ene-diol form.

Using the described BLAST assay method, three different sugar concentrations were tested in the BLAST assay: 2 mM (standard BLAST ascorbate concentration), 6 mM (3×), and 20 mM (10×). In addition to the normal copper sulfate concentration of 50 M, the four sugars were also tested using 200 μM (4×) copper sulfate. See TABLE 9, below.

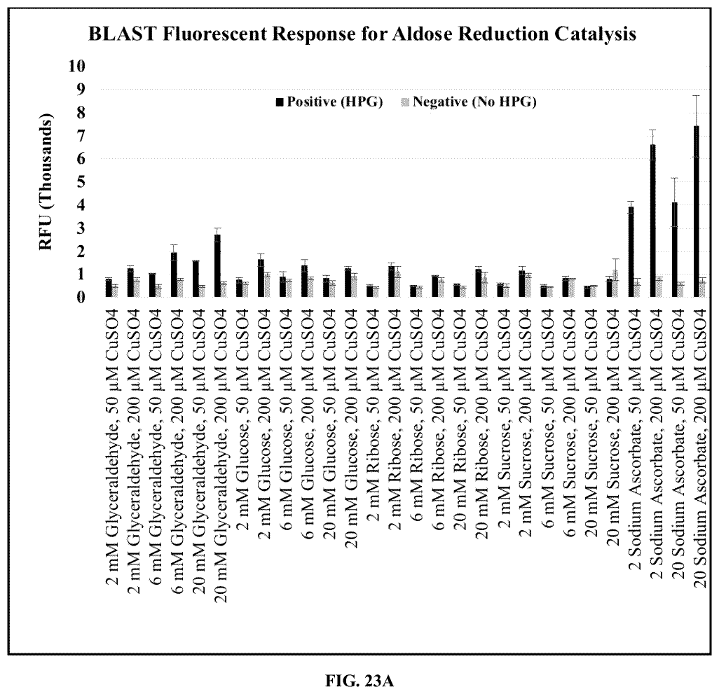

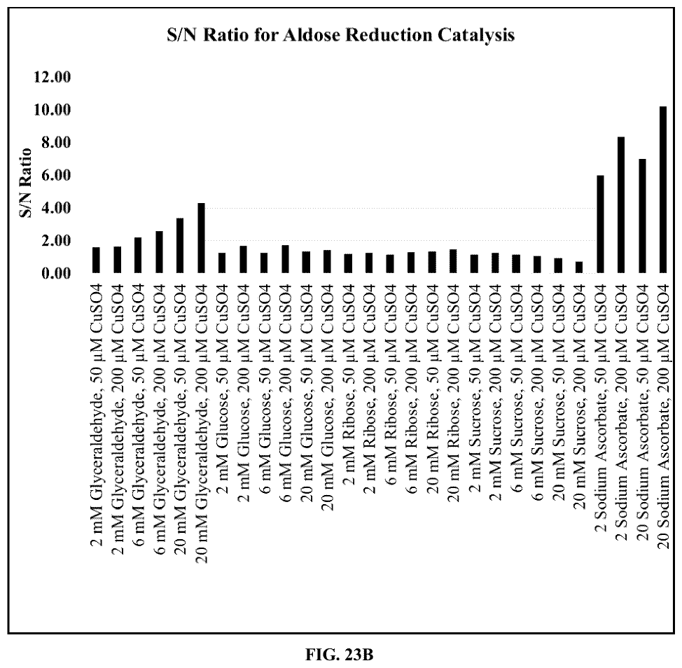

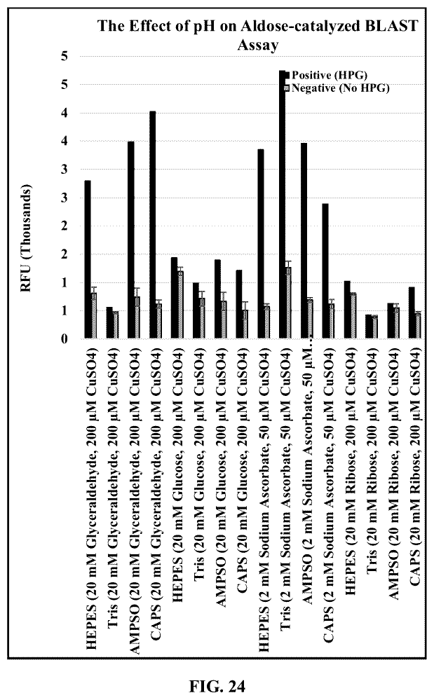

After the first study, a follow-up study was performed by testing the sugars in the BLAST assay at different pH conditions. These follow-up experiments were performed using the optimal sugar and copper concentrations determined in the first study. BLAST assays were performed with the sugars as well as with ascorbate at four different pH conditions as follows: pH 7.4 (normal conditions; HEPES buffer), pH 8 (Tris Buffer), pH 9 (TABS Buffer), and pH 10 (CAPS Buffer). All buffers contained NaCl and 0.1% Tween 20. In these experiments, all the buffers were substituted amines (Good's Buffers). However, other less expensive, high pH buffers such as borate or carbonate can be tested for future use. All tests were performed using E. coli ATCC 25922 at 5×10⁶ CFU/mL. The data shown in FIGS. 23A and 24 represent the average and standard deviations (n=3).

TABLE 9
Aldose reduction catalysis test plan.

| | | Test | |
|---|---|---|---|
| Reducing Agent | | Concentrations | |
| Type | Reducing Agent | of Sugar | CuSO₄ |
| Positive control | Sodium ascorbate | 2 mM (1X), 6 mM | 50 & |
| Test (Reducing | Glucose | (3X), & 20 mM | 200 uM |
| sugars) | Ribose | (10X) | |
| | Glyceraldehyde | | |
| Negative control | Sucrose (non- | | |
| | reducing sugar) | | |

Bacteria concentration: 5 × 10⁶ CFU/mL

| | |
|---|---|
| Click chemistry reaction time | 1 hour rather than 30 |
| | minutes (standard protocol) |

Standard concentration: Sodium ascorbate = 2 mM, CuSO₄ = 50 μM

In the first study, three sugars (glyceraldehyde, ribose and glucose) were tested in a BLAST assay using sucrose as the negative control and sodium ascorbate as the positive control. The results provided in FIGS. 23A and 23B show that 20 mM glyceraldehyde gave the best results when tested with 200 μM CuSO₄. Glyceraldehyde performed better because it is naturally acyclic. In the second study, the three sugars (glyceraldehyde, ribose and glucose) were tested in a BLAST assay using different pH (7.4, 8.0, 9.0, and 10) conditions with sodium ascorbate as the positive control. The results (see FIG. 24) show that glyceraldehyde gave the best results with pH 10 resulting in the highest signal. Glucose had less activity than ribose in the pH ranges and conditions tested. These results illustrate that reducing sugars can be used as an alternative to sodium ascorbate in the BLAST tagging process. Glyceraldehyde can be used in the assay with optimal tagging at pH 9 and 10 and is better than ribose and glucose. The performance of the three sugars can be optimized further by using different buffers.

As previously stated herein, the disclosed test method, assay, and test kit may be used to determine antimicrobial susceptibility in numerous different types of pathogenic or infectious microorganisms including, but not limited to, bacteria, mycoplasmas, yeasts, fungal pathogens, and protozoans. Test samples are described as “native” biological samples because the samples are taken directly from a patient, human or animal, and typically from the patient's bodily fluid(s) or in some cases, tissues. For fungal and other non-bacterial pathogens, a suitable acclimatization buffer (nutrient solution) is used to activate protein biosynthesis, for example YPD media may be used for yeast. YPD contains 20 g of Peptone, 10 g of yeast extract, 20 g of glucose in a sterile solution of 1 liter of water. Test samples may be taken directly from, or cultured from urine, blood, sputum, synovial fluid, cerebrospinal fluid, saliva, breast milk, wound discharge fluid, ascites, semen, vaginal discharge, nasal mucus, feces, or other fluids. The library of antimicrobials may include antibiotics, antifungals, bacteriophage (phage), or other drugs or compounds. Suitable classes of antibiotics include beta-lactams, tetracyclines, aminoglycosides, macrolides, fluoroquinolones, sulfonamides, glycopeptides, oxazolidinones, ansamycins, lipopeptides, streptogramins, lincosamides, polymyxins, or combinations thereof. Suitable classes of antifungals include azoles, echinocandins, polyenes, allylamines, flucytosine, griseofulvin, topical antifungals, or combinations thereof. As described herein, the non-canonical amino acid may be homopropargylglycine (HPG), which includes an alkyne moiety. The detectable element may be a fluorophore-tagged dye that includes an azide group that reacts with the alkyne moiety of HPG. The detectable element may be either an azide-modified biotin that reacts with the alkyne moiety of HPG, or an azido-conjugated enzyme that reacts with the alkyne moiety of HPG. Alternately, the non-canonical amino acid may be 3-Azido-L-alanine hydrochloride, which includes an azide group. The detectable element may be a fluorophore-tagged dye that includes an alkyne moiety that reacts with the azide group of 3-Azido-L-alanine hydrochloride.

All literature and similar material cited in this application, including, but not limited to, patents, patent applications, articles, books, treatises, and web pages, regardless of the format of such literature and similar materials, are expressly incorporated by reference in their entirety. Should one or more of the incorporated references and similar materials differ from or contradict this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about”, if or when used throughout this specification describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed technology. Various functions and elements described herein may be partitioned differently from those shown without departing from the scope of the disclosed technology. Generic principles defined herein may be applied to other implementations. Different numbers of a given module or unit may be employed, a different type or types of a given module or unit may be employed, a given module or unit may be added, or a given module or unit may be omitted.

Regarding this disclosure, the term “a plurality of” refers to two or more than two. Unless otherwise clearly defined, orientation or positional relations indicated by terms such as “upper” and “lower” are based on the orientation or positional relations as shown in the Figures, only for facilitating description of the disclosed technology and simplifying the description, rather than indicating or implying that the referred devices or elements must be in a particular orientation or constructed or operated in the particular orientation, and therefore they should not be construed as limiting the disclosed technology. The terms “connected”, “mounted”, “fixed”, etc. should be understood in a broad sense. For example, “connected” may be a fixed connection, a detachable connection, or an integral connection, a direct connection, or an indirect connection through an intermediate medium. For one of ordinary skill in the art, the specific meaning of the above terms in the disclosed technology may be understood according to specific circumstances.

The disclosed technology can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

It should be appreciated that all combinations of the foregoing concepts and additional concepts discussed in greater detail herein (provided such concepts are not mutually inconsistent) are contemplated as being part of the disclosed technology. In particular, all combinations of claimed subject matter appearing at the end of this disclosure are contemplated as being part of the technology disclosed herein. While the disclosed technology has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed technology in its broader aspects is not limited to any of the specific details, representative devices and methods, and/or illustrative examples shown and described. Accordingly, departures may be made from such details without departing from the spirit or scope of the general inventive concept.

The following references form part of the specification of the present application and each reference is incorporated by reference herein, in its entirety, for all purposes.
1. Antibacterial Susceptibility Test Interpretive Criteria|FDA.
2. Drew, K. N., Zajicek, J., Bondob, G., Bosea, B., and Serianni, A. S. (1998) 13C-labeled aldopentoses: detection and quantitation of cyclic and acyclic forms by heteronuclear 1D and 8 2D NMR spectroscopy. Carbohydrate Research 307, 199-209.
3. Guy, C. S., Tomás, R. M. F., Tang, Q., Gibson, M. I., & Fullam, E. (2022). Imaging of antitubercular dimeric boronic acids at the mycobacterial cell surface by click-probe capture. Chemical Communications, 58 (67), 9361-9364. https://doi.org/10.1039/d2cc02407a
4. Sarah, C., Andrew, J. W., Alexander, R. O., & Tamara, L. K. (2016). Incorporation of non-canonical amino acids into the developing murine proteome. Scientific Reports, 6, 32377. https://doi.org/10.1038/srep32377
5. Sherratt, A. R., Rouleau, Y., Luebbert, C., Strmiskova, M., Veres, T., Bidawid, S., Corneau, N. and Pezacki, J. P., 2017. Rapid screening and identification of living pathogenic organisms via optimized bioorthogonal non-canonical amino acid tagging. Cell Chemical Biology, 24 (8), pp. 1048-1055.
6. Singh, S. V., Saxena, and Singh, M. P. (1970) Mechanism of copper (II) oxidation of reducing sugars. I. Kinetics and mechanism of oxidation of D-xylose, L-arabinose, D-glucose, D-fructose, D-mannose, D-galactose, L-sorbose, lactose, maltose, cellobiose, and melibiose by copper (II) in alkaline medium. JACS 92, 537-541.
7. Zhu, Y., Zajicek, J. and Serianni, A. S. (2001) Acyclic Forms of [1-13C] Aldohexoses in Aqueous Solution: Quantitation by 13C NMR and Deuterium Isotope Effects on Tautomeric Equilibria. J. Org. Chem 66, 6244-6251.
8. Clinical and Laboratory Standards Institute. Performance standards for antimicrobial susceptibility testing. 33rd Approved standard, M100-S33, Clinical Laboratory and Standards Institute, Wayne, PA: CLSI; 2023.
9. Clinical and Laboratory Standards Institute. Methods for Dilution Antimicrobial Susceptibility Tests for Bacteria that Grow Aerobically; Approved Standard-11th Edition. CLSI document M07-A11. Wayne, PA: CLSI; 2018.

What is claimed:

1. A method for determining the susceptibility of microorganisms to various antimicrobials, comprising:
(a) activating protein biosynthesis in living microorganisms obtained from a native biological sample in an acclimatization buffer, wherein the acclimatization buffer is operative to activate the metabolism of the living microorganisms;
(b) exposing the living microorganisms to a library of antimicrobials,
(i) wherein the library of antimicrobials includes a plurality of antimicrobials at predetermined concentrations, and
(ii) wherein exposure either kills the microorganisms or blocks protein biosynthesis in the microorganisms that are sensitive to one or more of the antimicrobials at one or more of the predetermined concentrations;
(c) labeling newly biosynthesized proteins produced by the living microorganisms that survive exposure to the antimicrobials by incorporating a non-canonical amino acid into the biosynthesized proteins;
(d) tagging the labeled proteins with a detectable element by attaching the detectable element to the non-canonical amino acid, wherein tagging the labeled proteins with the detectable element creates an amount of detectable signal; and
(e) detecting the signal and comparing the amount of detected signal to a positive control,
(i) wherein an observed absence of or a decrease in the amount of detectable signal relative to the positive control indicates effectiveness of one or more of the antimicrobials in the library of antimicrobials against the living microorganisms at one or more of the predetermined concentrations; and
(ii) wherein an observed signal that approaches or is equal to the value of the positive control indicates ineffectiveness of one or more of the antimicrobials in the library of antimicrobials against the living microorganisms at one or more of the predetermined concentrations.

2. The method of claim 1, further comprising using the absence of or decrease in detectable signal at a particular concentration of an effective antimicrobial to determine a minimum inhibitory concentration for each effective antimicrobial in the library of antimicrobials.

3. The method of claim 1, further comprising using a wash buffer to remove any unincorporated non-canonical amino acid and using a wash buffer to remove any unattached detectable element, wherein one or both wash buffers contain a surfactant.

4. The method of claim 1, wherein the living microorganisms include bacteria.

5. The method of claim 1, wherein the living microorganisms include mycoplasmas, yeasts, fungal pathogens, protozoans, or combinations thereof.

6. The method of claim 1, wherein the native biological sample includes homogenized biopsy material, and wherein the homogenized biopsy material includes muscle, skin, or internal organs.

7. The method of claim 1, wherein the native biological sample is taken directly from a bodily fluid, or wherein the native biological sample is an isolated colony cultured from a bodily fluid.

8. The method of claim 7, wherein the bodily fluid is urine.

9. The method of claim 7, wherein the bodily fluid is blood, sputum, synovial fluid, cerebrospinal fluid, saliva, breast milk, wound discharge fluid, ascites, semen, vaginal discharge, nasal mucus, or feces.

10. The method of claim 1, wherein the library of antimicrobials includes antibiotics, antifungals, or a combination thereof.

11. The method of claim 10,
(a) wherein the antibiotics include beta-lactams, tetracyclines, aminoglycosides, macrolides, fluoroquinolones, sulfonamides, glycopeptides, oxazolidinones, ansamycins, lipopeptides, streptogramins, lincosamides, polymyxins, or combinations thereof; and
(b) wherein the antifungals include azoles, echinocandins, polyenes, allylamines, flucytosine, griseofulvin, topical antifungals, or combinations thereof.

12. The method of claim 1, wherein the library of antimicrobials includes bacteriophage.

13. The method of claim 1, wherein the non-canonical amino acid is homopropargylglycine (HPG), wherein the HPG includes an alkyne moiety, and wherein the newly biosynthesized proteins include the alkyne moiety.

14. The method of claim 13, wherein the detectable element is a fluorophore-tagged dye, and wherein the fluorophore-tagged dye includes an azide group that reacts with the alkyne moiety of HPG.

15. The method of claim 13, wherein the detectable element is an azide-modified biotin that reacts with the alkyne moiety of HPG or an azido-conjugated enzyme that reacts with the alkyne moiety of HPG.

16. The method of claim 1, wherein the non-canonical amino acid is 3-Azido-L-alanine hydrochloride, wherein the 3-Azido-L-alanine hydrochloride includes an azide group, and wherein the newly biosynthesized proteins include the azide group.

17. The method of claim 16, wherein the detectable element is a fluorophore-tagged dye, and wherein the fluorophore-tagged dye includes an alkyne moiety that reacts with the azide group of 3-Azido-L-alanine hydrochloride.

18. The method of claim 1, wherein the attachment of the detectable element to the labeled protein is accomplished using a copper catalysis that includes Copper I ions and a stabilizing ligand.

19. The method of claim 18, wherein the copper catalysis is activated by addition of a reducing agent to a mixture of copper II ions and the stabilizing ligand, and wherein the reducing agent is ascorbic acid, glyceraldehyde, or another reducing sugar.

20. The method of claim 1, further comprising arranging reagents used in the method in a kit, wherein the kit includes lyophilized buffers and lyophilized antimicrobials exhibiting prolonged shelf-life.

21. A test method for determining the susceptibility of microorganisms to various antimicrobials, comprising:
(a) activating protein biosynthesis in living microorganisms obtained from an uncultured native biological sample taken directly from a bodily fluid in an acclimatization buffer, wherein the acclimatization buffer is operative to activate the metabolism of the living microorganisms;
(b) exposing the living microorganisms to a library of antimicrobials,
(i) wherein the library of antimicrobials includes a plurality of antimicrobials at predetermined concentrations, and
(ii) wherein exposure either kills the microorganisms or blocks protein biosynthesis in the microorganisms that are sensitive to one or more of the antimicrobials at one or more of the predetermined concentrations;
(c) labeling newly biosynthesized proteins produced by the living microorganisms that survive exposure to the antimicrobials by incorporating a non-canonical amino acid into the biosynthesized proteins;
(d) tagging the labeled proteins with a detectable element by attaching the detectable element to the non-canonical amino acid, wherein tagging the labeled proteins with the detectable element creates an amount of detectable signal;
(e) detecting the signal and comparing the amount of detected signal to a positive control,
(i) wherein an observed absence of or a decrease in the amount of detectable signal relative to the positive control indicates effectiveness of one or more of the antimicrobials in the library of antimicrobials against the living microorganisms at one or more of the predetermined concentrations; and
(ii) wherein an observed signal that approaches or is equal to the value of the positive control indicates ineffectiveness of one or more of the antimicrobials in the library of antimicrobials against the living microorganisms at one or more of the predetermined concentrations; and
(f) using the absence of or decrease in detectable signal at a particular concentration of an effective antimicrobial to determine a minimum inhibitory concentration for each effective antimicrobial in the library of antimicrobials.

22. The method of claim 21, further comprising using a wash buffer to remove any unincorporated non-canonical amino acid and using a wash buffer to remove any unattached detectable element, wherein one or both wash buffers contain a surfactant.

23. The method of claim 21, wherein the living microorganisms include bacteria, mycoplasmas, yeasts, fungal pathogens, protozoans, or combinations thereof.

24. The method of claim 21, wherein the native biological sample includes homogenized biopsy material, and wherein the homogenized biopsy material includes muscle, skin, or internal organs.

25. The method of claim 21, wherein the native biological sample is taken directly from a bodily fluid, or wherein the native biological sample is an isolated colony cultured from a bodily fluid.

26. The method of claim 25, wherein the bodily fluid is urine, blood, sputum, synovial fluid, cerebrospinal fluid, saliva, breast milk, wound discharge fluid, ascites, semen, vaginal discharge, nasal mucus, or feces.

27. The method of claim 21, wherein the library of antimicrobials includes antibiotics, antifungals, or a combination thereof,
(a) wherein the antibiotics include beta-lactams, tetracyclines, aminoglycosides, macrolides, fluoroquinolones, sulfonamides, glycopeptides, oxazolidinones, ansamycins, lipopeptides, streptogramins, lincosamides, polymyxins, or combinations thereof; and
(b) wherein the antifungals include azoles, echinocandins, polyenes, allylamines, flucytosine, griseofulvin, topical antifungals, or combinations thereof.

28. The method of claim 21, wherein the library of antimicrobials includes bacteriophage.

29. The method of claim 21, wherein the non-canonical amino acid is homopropargylglycine (HPG), wherein the HPG includes an alkyne moiety, and wherein the newly biosynthesized proteins include the alkyne moiety.

30. The method of claim 21,
(a) wherein the detectable element is a fluorophore-tagged dye, and wherein the fluorophore-tagged dye includes an azide group that reacts with the alkyne moiety of HPG, or
(b) wherein the detectable element is an azide-modified biotin that reacts with the alkyne moiety of HPG or an azido-conjugated enzyme that reacts with the alkyne moiety of HPG.

31. The method of claim 21, wherein the non-canonical amino acid is 3-Azido-L-alanine hydrochloride, wherein the 3-Azido-L-alanine hydrochloride includes an azide group, and wherein the newly biosynthesized proteins include the azide group.

32. The method of claim 31, wherein the detectable element is a fluorophore-tagged dye, and wherein the fluorophore-tagged dye includes an alkyne moiety that reacts with the azide group of 3-Azido-L-alanine hydrochloride.

33. The method of claim 21, wherein the attachment of the detectable element to the labeled protein is accomplished using a copper catalysis that includes Copper I ions and a stabilizing ligand.

34. The method of claim 33, wherein the copper catalysis is activated by addition of a reducing agent to a mixture of copper II ions and the stabilizing ligand, and wherein the reducing agent is ascorbic acid, glyceraldehyde, or another reducing sugar.

35. The method of claim 21, further comprising arranging reagents used in the test method in a kit, wherein the kit includes lyophilized buffers and lyophilized antimicrobials exhibiting prolonged shelf-life.

36. A test method for determining the susceptibility of microorganisms to various antimicrobials, comprising:
(a) activating protein biosynthesis in living microorganisms obtained from either an uncultured native biological sample taken directly from a bodily fluid or an isolated colony cultured from a bodily fluid in an acclimatization buffer for a predetermined period of time, wherein the acclimatization buffer is operative to activate the metabolism of the living microorganisms;
(b) exposing the living microorganisms to a library of antimicrobials for a predetermined period of time;
(i) wherein the library of antimicrobials includes a plurality of antimicrobials at predetermined concentrations, and
(ii) wherein exposure either kills the microorganisms or blocks protein biosynthesis in the microorganisms that are sensitive to one or more of the antimicrobials at one or more of the predetermined concentrations;
(c) labeling newly biosynthesized proteins produced by the living microorganisms that survive exposure to the antimicrobials by incorporating a non-canonical amino acid into the biosynthesized proteins;
(d) tagging the labeled proteins with a detectable element by attaching the detectable element to the non-canonical amino acid, wherein tagging the labeled proteins with the detectable element creates an amount of detectable signal;
(e) detecting the signal and comparing the amount of detected signal to a positive control,
(i) wherein an observed absence of or a decrease in the amount of detectable signal relative to the positive control indicates effectiveness of one or more of the antimicrobials in the library of antimicrobials against the living microorganisms at one or more of the predetermined concentrations; and
(ii) wherein an observed signal that approaches or is equal to the value of the positive control indicates ineffectiveness of one or more of the antimicrobials in the library of antimicrobials against the living microorganisms at one or more of the predetermined concentrations; and
(f) using the absence of or decrease in detectable signal at a particular concentration of an effective antimicrobial to determine a minimum inhibitory concentration for each effective antimicrobial in the library of antimicrobials.

37. The method of claim 36, further comprising using a wash buffer to remove any unincorporated non-canonical amino acid and using a wash buffer to remove any unattached detectable element, wherein one or both wash buffers contain a surfactant.

38. The method of claim 36, wherein the living microorganisms include bacteria, mycoplasmas, yeasts, fungal pathogens, protozoans, or combinations thereof.

39. The method of claim 36, wherein the native biological sample includes homogenized biopsy material, and wherein the homogenized biopsy material includes muscle, skin, or internal organs.

40. The method of claim 36, wherein the native biological sample is taken directly from a bodily fluid, or wherein the native biological sample is an isolated colony cultured from a bodily fluid, and wherein the bodily fluid is urine, blood, sputum, synovial fluid, cerebrospinal fluid, saliva, breast milk, wound discharge fluid, ascites, semen, vaginal discharge, nasal mucus, or feces.

41. The method of claim 36, wherein the library of antimicrobials includes antibiotics, antifungals, or a combination thereof,
(a) wherein the antibiotics include beta-lactams, tetracyclines, aminoglycosides, macrolides, fluoroquinolones, sulfonamides, glycopeptides, oxazolidinones, ansamycins, lipopeptides, streptogramins, lincosamides, polymyxins, or combinations thereof; and
(b) wherein the antifungals include azoles, echinocandins, polyenes, allylamines, flucytosine, griseofulvin, topical antifungals, or combinations thereof.

42. The method of claim 36, wherein the library of antimicrobials includes bacteriophage.

43. The method of claim 36, wherein the non-canonical amino acid is homopropargylglycine (HPG), wherein the HPG includes an alkyne moiety, and wherein the newly biosynthesized proteins include the alkyne moiety.

44. The method of claim 36,
(a) wherein the detectable element is a fluorophore-tagged dye, and wherein the fluorophore-tagged dye includes an azide group that reacts with the alkyne moiety of HPG, or
(b) wherein the detectable element is an azide-modified biotin that reacts with the alkyne moiety of HPG or an azido-conjugated enzyme that reacts with the alkyne moiety of HPG.

45. The method of claim 36, wherein the non-canonical amino acid is 3-Azido-L-alanine hydrochloride, wherein the 3-Azido-L-alanine hydrochloride includes an azide group, and wherein the newly biosynthesized proteins include the azide group.

46. The method of claim 41, wherein the detectable element is a fluorophore-tagged dye, and wherein the fluorophore-tagged dye includes an alkyne moiety that reacts with the azide group of 3-Azido-L-alanine hydrochloride.

47. The method of claim 36, wherein the attachment of the detectable element to the labeled protein is accomplished using a copper catalysis that includes Copper I ions and a stabilizing ligand.

48. The method of claim 47, wherein the copper catalysis is activated by addition of a reducing agent to a mixture of copper II ions and the stabilizing ligand, and wherein the reducing agent is ascorbic acid, glyceraldehyde, or another reducing sugar.

49. The method of claim 36, further comprising arranging reagents used in the test method in a kit, wherein the kit includes lyophilized buffers and lyophilized antimicrobials exhibiting prolonged shelf-life.
